(19) United States
(12) Patent Application Publication
(10) Pub. No.: US 2024/0001019 A1
(43) Pub. Date: Jan. 4, 2024

(54) MBL-COATED SUBSTRATES HAVING ANTI-THROMBOGENIC PROPERTIES

(71) Applicant: MIRAKI INNOVATION THINK TANK LLC, Cambridge, MA (US)

(72) Inventors: Nisha Veronica VARMA, Brookline, MA (US); Zhiqian ZHOU, Boston, MA (US); Keith CRAWFORD, Westwood, MA (US); Andrew D. CONNERTY, Boxford, MA (US); Shailendra KUMAR, Needham, MA (US)

(73) Assignee: MIRAKI INNOVATION THINK TANK LLC, Cambridge, MA (US)

(21) Appl. No.: 18/038,347

(22) PCT Filed: Jul. 30, 2021
(86) PCT No.: PCT/US21/43849

§ 371 Date: May 23, 2023

Related U.S. Application Data
(60) Provisional application No. 63/059,485, filed on Jul. 31, 2020.

(51) Int. Cl. A61M 1/36; A61L 33/00; A61L 31/10; C07K 14/705

(52) U.S. Cl. CPC A61M 1/3687; A61L 33/0011; A61L 31/10; C07K 14/7056; C07K 2319/30

(57) ABSTRACT

The present invention provides mannose-binding lectin (MBL)-based compositions, and methods of using the MBL-based compositions in the reduction of unintended platelet activation, such as during a medical procedure.

### SEQUENCE LISTING

A sequence listing in electronic (ASCII text file) format is filed with this application and incorporated herein by reference. The name of the ASCII text file is “2021_1208A_ST25.txt”; the file was created on Jul. 30, 2021; the size of the file is 40 KB.

### FIELD OF THE INVENTION

The present invention relates to methods and means for reducing unintended platelet activation in the blood, such as in the blood of a subject undergoing a medical procedure.

### BACKGROUND

Extracorporeal membrane oxygenation (ECMO), also known as extracorporeal life support (ECLS), is a medical technique used to supplement and support cardiac and respiratory functions in a patient. The use of ECMO allows extracorporeal oxygenation of the blood in a patient whose heart and/or lungs are otherwise unable to provide sufficient gas exchange to sustain life.

The technique directs blood out of the body and into a device that permits concomitant oxygenation of the red blood cells and removal of carbon dioxide. As gas exchange is achieved, the blood is directed back into the body of the patent. ECMO is commonly employed during patient recovery after cardiopulmonary bypass and in late-stage treatment of profound heart and/or lung failure. However, the technique also sees use in any medical treatment or application where there is a reduction or cessation of normal heart and/or lung function, whether temporary or permanent, while circulation and oxygenation are supported. Recent reports also suggest ECMO can be used to support patients with serious respiratory failure often associated with COVID-19, such as in those patients where artificial ventilation is not sufficient to sustain necessary levels of blood oxygenation.

While ECMO is an important, life-saving medical technology, it does have at least one potentially serious side effect. During its passage outside of the body, the circulating blood comes into contact with artificial surfaces that may trigger unintended activation of platelets. If not controlled, platelet activation could lead to clot formation (thrombosis), and in turn, formation of an embolism. Also, in light of the depletion of platelets from the blood, excessive and/or uncontrolled bleeding is a common complication associated with the use of ECMO, where 23% of patients undergoing ECMO suffered severe bleeding.

In addition, infections are more prone to occur when a cannula is inserted into a blood vessel, a requirement of ECMO. Bloodstream infection is one of the most common complications, occurring in ˜14.4% of patients on ECMO. Longer hospital stays (more than 15 days) increases chances of contracting hospital-acquired infections.

In addition to the depletion of platelets from the blood of patients undergoing ECMO, reports have demonstrated that the recognition molecule ficolin-2 of the lectin pathway is depleted from plasma when heparin-coated circuits are used, and such circuits may be used in ECMO procedures. The depletion of this molecule could lead to increased susceptibility to infections in patients undergoing extracorporeal circulation procedures. It is reasonable to speculate that the partial depletion of ficolin-2 could have a significant impact on patient health and outcome, at least during specific disease settings or in immune-compromised individuals. This is especially likely given the spectrum of opportunistic microorganisms that ficolin-2 has been reported to recognize: capsulated Staphylococcus aureus, Streptococcus pneumonia, Salmonella typhimurium, Escherichia coli, Pseudomonas aeruginosa and Aspergillus fumigatus.

Similarly, reports have demonstrated that depletion of antibiotics from the blood of patients undergoing extracorporeal procedures, such as ECMO, can occur. It has been shown that in some cases, antibiotics, such as glycoproteins, can adhere to components such as filters and membranes used in such procedures, when antibiotics have been administered to a subject, either before or during such procedures.

Thus, there is an unmet need for means to reduce unintended platelet activation, clotting and bleeding, as well as means to reduce depletion of molecules such as ficolin-2 and antibiotics from the blood, in patients undergoing extracorporeal procedures, such as ECMO.

### SUMMARY

Briefly, the present invention is based on the discovery that when filters used in extracorporeal therapy devices are coated with a mannose-binding lectin (MBL), there is a decrease in unintended platelet activation and coagulation in the blood contacting the filters. Thus, the MBL-coated filters of the present invention protect against unintended platelet activation, coagulation and thrombocytopenia (low platelet count) in a subject. As a result, the filters also decrease complications associated with bleeding (due to platelet depletion) and they possess antithrombogenic properties.

In addition, use of the MBL-coated filters of the invention avoids unintended depletion of antibiotics from the blood. Thus, the MBL-coated substrates of the present invention also protect against antibiotic depletion. The MBL-coated substrates may further protect against ficolin-2 depletion, and they may be of significant benefit during hemodialysis or oxygenation of blood in MBL-deficient patients. The MBL-coated substrates may be used in subjects known to be MBL-deficient.

Mannose-binding lectin (MBL) is a member of the collectin family of lectins. The activity and attributes of MBL disclosed herein may also be exhibited by other lectins within the collectin family. Thus, the invention is generally directed to (i) coatings or films comprising a collectin, (ii) substrates coated with a collectin, such as filters and components of medical devices, (iii) methods for reducing platelet activation in blood using the coatings, films and substrates of the invention, and (iv) methods for reducing platelet activation in a subject undergoing a medical procedure using the coatings, films and substrates of the invention. In each aspect and embodiment of the invention, the collectin may be, but is not limited to, MBL, whether naturally-occurring or engineered (e.g. FcMBL).

Thus, and in a first embodiment, the present invention is directed to a substrate having at least one surface coated with one or more collectins.

The collectin may be selected from (i) mannose-binding lectin (MBL), (ii) surfactant protein A (SP-A), (iii) surfactant protein D (SP-D), (iv) collectin liver 1 (CL-L1), (v) collectin placenta 1 (CL-P1), and (vi) collectin kidney 1 (CL-K1).

In one aspect of this embodiment, the collectin is (i) a naturally-occurring MBL, (ii) a truncated form of naturally-occurring MBL, (iii) an engineered form of MBL, or (iv) a sequence variant of (i), (ii) or (iii).

In a specific aspect of this embodiment, the collectin is a naturally-occurring MBL as set forth in SEQ ID NO:1 or a sequence variant having at least 80% sequence identity with SEQ ID NO:1 that retains the ability to reduce platelet activation in blood.

In another specific aspect of this embodiment, the collectin is a truncated form of naturally-occurring MBL as set forth in any one of SEQ ID NOs:2-5 or a sequence variant having at least 80% sequence identity with any one of SEQ ID NOs:2-5 that retains the ability to reduce platelet activation in blood.

In a further specific aspect of this embodiment, the collectin is an engineered form of MBL comprising a fusion protein of an MBL of any one of SEQ ID NOs:1-5 and an Fc domain of an IgG antibody. For example, the fusion protein may be a FcMBL protein set forth in any one of SEQ ID NOs:6-9, or a sequence variant having at least 80% sequence identity with any one of SEQ ID NOs:6-9 that retains the ability to reduce platelet activation in blood.

The collectins may be immobilized on the surface via a non-specific interaction between the collectins and the surface. Alternatively, the collectins may be immobilized on the surface via a specific interaction between the collectins and the surface. For example, 3-(3-dimethylaminopropyl)carbodiimide (EDC) may be used to create a covalent amide bond between the collectin and the surface to which it is attached. The specific interaction may also be a linker that is immobilized on the surface and that exhibits binding specificity for the collectin.

In certain aspects of this embodiment, the surface is fabricated of or coated with one or more of following prior to coating of the surface with the collectins: polydimethylsiloxane, polyimide, polyethylene terephthalate, polymethylmethacrylate, polyurethane, polyvinylchloride, polystyrene polysulfone, polycarbonate, polymethylpentene, polypropylene, a polyvinylidine fluoride, polysilicon, polytetrafluoroethylene, polysulfone, acrylonitrile butadiene styrene, polyacrylonitrile, polybutadiene, poly(butylene terephthalate), poly(ether sulfone), poly(ether ether ketones), poly(ethylene glycol), styrene-acrylonitrile resin, poly(trimethylene terephthalate), polyvinyl butyral, polyvinylidenedifluoride, poly(vinyl pyrrolidone), and any combination thereof.

In certain aspects of this embodiment, the surface is functionalized to include at least one molecule exhibiting binding specificity for the collectin prior to coating of the surface with the one or more collectins.

In certain aspects of this embodiment, the surface is also coated with an anticoagulant. Suitable anticoagulants include, but are not limited to, heparin, warfarin, rivaroxaban, dabigatran, apixaban, edoxaban, enoxaparin, or and fondaparinux.

In aspects of this embodiment, the substrate may be one or more of a bead, a particle, a filter, a fiber, a screen, a mesh, a membrane, a semi-permeable membrane, a permeable membrane, a cartridge, a tube, a hollow fiber, a cell-culture scaffold, a cell-culture plate, a channel, a gold particle, a magnetic material, a needle, a catheter, a syringe, a medical or dental appliance, a medical or dental implant, a medical component, a dialyzer, a hemodialyzer, a dipstick, a test strip, a filtration device, a filtration device membrane, a hollow fiber cartridge, a microfluidic device, a mixing element, a component of a medical device, a component of a therapeutic device, a filtration device, an oxygenation device, an extracorporeal device, a dialysis device, an infusion device, a drainage device, and a pump.

In a specific aspect of this embodiment, the substrate is a filter, such as a filter of an extracorporeal therapy device such as an extracorporeal membrane oxygenation (ECMO) device.

In a second embodiment, the invention is directed to methods for reducing platelet activation in a subject undergoing a medical procedure. The method comprises conducting a medical procedure on the subject using a medical device comprising at least one substrate as defined herein.

In a related embodiment, the invention is directed to methods for reducing platelet activation in a subject undergoing an extracorporeal therapy such as extracorporeal membrane oxygenation (ECMO). The method comprises conducting an extracorporeal therapy such as ECMO on the subject using a device comprising at least one substrate as defined herein.

In a further related embodiment, the invention is directed to methods for reducing platelet activation in blood of a subject undergoing a medical procedure. The method comprises contacting blood of the subject using a device comprising at least one substrate as defined herein. As an example, the medical procedure may be oxygenation of the blood, or removal of carbon dioxide from the blood, or both.

In a third embodiment, the invention is directed to methods for reducing antibiotic loss from blood of a subject undergoing a medical procedure. The method comprises conducting a medical procedure on the subject using a medical device comprising at least one substrate as defined herein.

In a related embodiment, the invention is directed to methods for reducing antibiotic loss from blood of a subject undergoing an extracorporeal therapy such as extracorporeal membrane oxygenation (ECMO). The method comprises conducting an extracorporeal therapy such as ECMO on the subject using a device comprising at least one substrate as defined herein.

In a fourth embodiment, the invention is directed to methods for reducing ficolin-2 loss from blood of a subject undergoing a medical procedure. The method comprises conducting a medical procedure on the subject using a medical device comprising at least one substrate as defined herein.

In a related embodiment, the invention is directed to methods for reducing ficolin-2 loss from blood of a subject undergoing an extracorporeal therapy such as extracorporeal membrane oxygenation (ECMO). The method comprises conducting an extracorporeal therapy such as ECMO on the subject using a device comprising at least one substrate as defined herein.

### BRIEF DESCRIPTION OF THE DRAWINGS

FIG. 1: Structural features of mannose-binding lectin (MBL).

FIG. 2: WBC as a function of time for Filtriva versus Revaclear 400.

FIG. 3: RBC as a function of time for Filtriva versus Revaclear 400.

FIG. 4: PC as a function of time for Filtriva versus Revaclear 400.

FIG. 5: PF4 as a function of time for Filtriva versus Revaclear 400.

FIG. 6: CD62P as a function of time for Filtriva versus Revaclear 400.

FIG. 7: TAT as a function of time for Filtriva versus Revaclear 400.

FIG. 8: Heparin as a function of time for Filtriva versus Revaclear 400.

### DETAILED DESCRIPTION

Collectins (collagen-containing C-type lectins) are a family of collagenous calcium-dependent lectins that function in defense, thus playing an important role in the innate immune system. They are soluble molecules comprising pattern recognition receptors (PRRs) within a microbe-binding domain that recognize and bind to particular oligosaccharide structures or lipids displayed on the surface of microbes, i.e. MAMPs (microbe-associated molecular patterns) of oligosaccharide origin. Upon binding of a collectin to a microbe, clearance of the microbe is achieved via aggregation, complement activation, opsonization, and activation of phagocytosis.

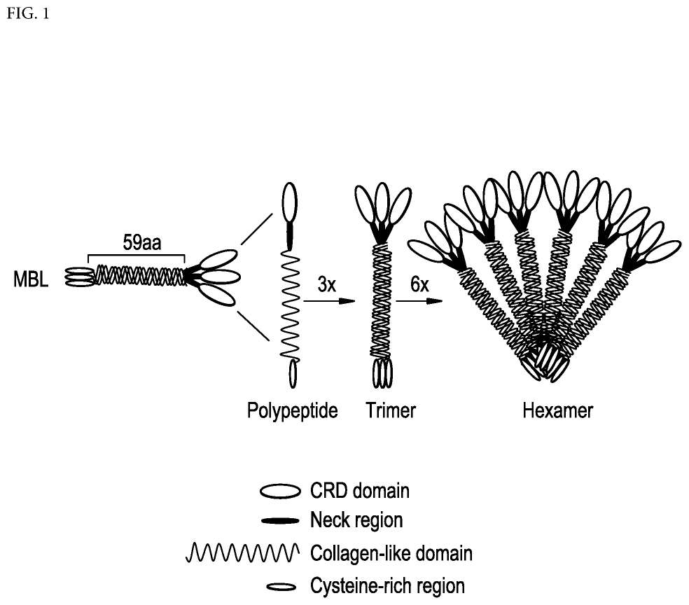

Members of the family have a common monomeric structure, characterized by four parts or domains arranged in the following N- to C-terminal arrangement: (i) a cysteine-rich region, (ii) a collagen-like domain, (iii) a coiled-coil neck region, and (iv) a microbe-binding domain which includes a C-type lectin domain, also termed the carbohydrate recognition domain (CRD) (FIG. 1). The functional form of the molecule is a trimer made up of three identical chains. MAMP recognition is mediated by the CRD in presence of calcium.

There are currently nine recognized members of the collectin family: (i) mannose-binding lectin (MBL; SEQ ID NO:1), (ii) surfactant protein A (SP-A), (iii) surfactant protein D (SP-D), (iv) collectin liver 1 (CL-L1), (v) collectin placenta 1 (CL-P1), (vi) conglutinin collectin of 43 kDa (CL-43), (vii) collectin of 46 kDa (CL-46), (viii) collectin kidney 1 (CL-K1), and (ix) conglutinin.

Mannose-binding lectin (MBL), also called mannan-binding lectin or mannan-binding protein (MBP), is an abundant mammalian serum protein (also found in plants). MBL binds to mannose, N-acetylglucosamine (NAG)-containing carbohydrates, and various other carbohydrates that are present on the surface of virtually all classes of pathogens (viruses, bacteria, fungi, protozoans). Upon binding of MBL to a microorganism or portion thereof, the lectin pathway of the complement system is activated.

MBL exists as a polymeric protein having an oligomeric structure (400-700 kDa) assembled from groups of three or more identical 32 kDa monomers. Each monomer has the four distinct regions mentioned above, namely: (i) a cysteine-rich N-terminal region, (ii) a collagen-like Gly-X-Y domain, (iii) a neck region comprising a short alpha-helical coiled coil domain, and (iv) a carbohydrate recognition domain (CRD). Three of the monomers form a triple helix through the collagenous region, which is stabilized via hydrophobic interaction and interchain disulfide bonds within the N-terminal cysteine-rich region. The higher molecular weight polymers of mature MBLs are assemblies of three to six sets of trimers [1] (FIG. 1).

MBL is produced by the liver, and the protein is often expressed in the serum under stress conditions with significantly increased circulating levels seen in response to infection. MBL activates macrophages, enhances phagocytosis [2, 3], and the protein has a role in complement activation by inducing the antibody-independent lectin pathway [4, 5, 6, 7-11]. In particular, MBL initiates the lectin pathway of complement activation, the release of cytokines, and coagulation factors, in conjunction with three MBL-associated serine proteases (MASP 1, 2, and 3), leading to cleavage of complement proteins C4, C2, and C3 [11, 12].

The ability of MBL to bind to surface molecules on virtually all classes of pathogens (viruses, bacteria, fungi, protozoans), as well as nucleic acids and exosomes (i.e. infectious and cancerous), makes the protein (and engineered forms thereof) extremely useful in diagnosing and treating infectious diseases and sepsis. Engineered forms of MBLs are known in the art and include each of the forms of MBL disclosed in U.S. Pat. No. 9,150,631, U.S. Patent Pub. 2016/0311877, U.S. Patent Pub. 2019/0077850, U.S. Pat. Nos. 9,593,160, 10,435,457, U.S. Patent Pub. 2015/0173883 and International Application Publication No. WO 2011/090954, the entire disclosures of each of which are hereby incorporated by reference in their entirety.

To use the proteins in practical applications, they can be coated onto a wide variety of surfaces, such a laboratory plates, beads, plasmonic surfaces, microchips, slides, and filters. Filters can be very useful because blood and other fluids can flow through the filter and when MBL is present, e.g. on the surface of the filter, the pathogen is bound to the filter, thus providing a diagnostic technique or even a means for clearing the micro-organism from the blood or fluid.

In conjunction with the preparation and testing of MBL-coated filters, the present inventors made the surprising discovery that not only were micro-organisms isolated from the blood and bound to the surface of the filters, there was a clear reduction in the amount of platelet activation and coagulation in blood exposed to the MBL-coated filters in comparison to un-coated filters. In addition, there did not appear to be a depletion in the amount of antibiotics present in the circulation after blood containing antibiotics was exposed to MBL-coated filters. Further, depletion in the amount of ficolin-2 levels present in the circulation after blood is exposed to MBL-coated filters may be reduced. Thus, filters coated with MBL protect against (i) unintended platelet activation and coagulation, (ii) thrombocytopenia, (iii) bleeding (due to platelet depletion), (iv) antibiotic depletion (optimized antibiotic clearance), and (v) ficolin-2 depletion, in comparison to uncoated versions of the same filters. In light of this discovery, coatings comprising collectins, such as MBLs, may be applied to any item that comes into contact with blood (e.g., filters, catheters, extracorporeal devices such as ECMO and/or oxygenation devices, etc.) as a means for decreasing undesired platelet activation in the blood, and without adverse effects on the circulation of antibiotics and/or ficolin-2 when they are present in the blood.

Problems solved by the present invention include decreasing and/or eliminating the clotting effect that can result from unintended activation of platelets during medical procedures, such as when circulating blood comes into contact with an artificial surface, such as the surface of a filter or a medical device. Problems solved by the present invention also include (i) complications associated with bleeding (due to platelet depletion), and (ii) decreasing and/or eliminating the depletion of antibiotics and/or ficolin-2 when circulating blood comes into contact with an artificial surface, such as the surface of a filter or a medical device, to which the antibiotics and/or ficolin-2 might otherwise bind.

Advantages of the present invention include reducing and/or preventing the clotting effect that results from unintended activation of platelets that can occur when circulating blood comes into contact with an artificial surface. For example, bleeding is the most common complication associated with the use of ECMO, and reducing platelet depletion results in decreased complications associated with bleeding. Advantages of the present invention also include reducing and/or preventing depletion of antibiotics and/or ficolin-2 from the blood when circulating blood in comes into contact with an artificial surface to which the antibiotics and/or ficolin-2 might otherwise bind.

### MBL

In each aspect and embodiment of the invention, any MBL, whether naturally-occurring or engineered, that reduces the amount of platelet activation in the blood, when used in conjunction with the methods and components of the invention (e.g., coatings, films and substrates), may be used in the aspects and embodiments of the invention as defined herein. Thus, as used herein, and unless the context indicates otherwise, the term “MBL” is intended to broadly reference both naturally-occurring MBL and engineered forms of the protein. As discussed below, engineered forms of the protein include, but are not limited to, truncated forms of the naturally-occurring protein, sequence variants of the naturally-occurring proteins, sequence variants of the truncated forms of the proteins, fusion proteins comprising the naturally-occurring protein, fusion proteins comprising the truncated forms of the proteins, and fusion proteins comprising the sequence variants.

In each aspect and embodiment of the invention, MBL may be full-length human MBL (SEQ ID NO:1), mature human MBL without the signal sequence (e.g. SEQ ID NO:2), a truncated human MBL that retains microbe surface-binding (e.g. SEQ ID NO:3), the carbohydrate recognition domain (CRD) of human MBL (e.g. SEQ ID NO:4), or the neck and carbohydrate recognition domain of human MBL (e.g. SEQ ID NO:5), whether used alone or in combination with a second protein in the form of a fusion protein, such as a FcMBL protein as defined herein.

The amino acid sequence of full-length human MBL (SEQ ID NO:1; GenBank: AAH69338.1) is:

MSLFPSLPLL LLSMVAASYS ETVTCEDAQK TCPAVIACSS
PGINGFPGKD GRDGTKGEKG EPGQGLRGLQ GPPGKLGPPG
NPGPSGSPGP KGQKGDPGKS PDGDSSLAAS ERKALQTEMA
RIKKWLTFSL GKQVGNKFFL TNGEIMTFEK VKALCVKFQA
SVATPRNAAE NGAIQNLIKE EAFLGITDEK TEGQFVDLTG
NRLTYTNWNE GEPNNAGSDE DCVLLLKNGQ WNDVPCSTSH
LAVCEFPI

The amino acid sequence of mature human MBL without the signal sequence (SEQ ID NO:2) is:

ETVTCEDAQK TCPAVIACSS PGINGFPGKD GRDGTKGEKG
EPGQGLRGLQ GPPGKLGPPG NPGPSGSPGP KGQKGDPGKS
PDGDSSLAAS ERKALQTEMA RIKKWLTFSL GKQVGNKFFL
TNGEIMTFEK VKALCVKFQA SVATPRNAAE NGAIQNLIKE
EAFLGITDEK TEGQFVDLTG NRLTYTNWNE GEPNNAGSDE
DCVLLLKNGQ WNDVPCSTSH LAVCEFPI

The amino acid sequence of a truncated MBL that retains microbe surface-binding (SEQ ID NO:3) is:

AASERKALQT EMARIKKWLT FSLGKQVGNK FFLINGEIMT
FEKVKALCVK FQASVATPRN AAENGAIQNL IKEEAFLGIT
DEKTEGQFVD LTGNRLTYTN WNEGEPNNAG SDEDCVLLLK
NGQWNDVPCS TSHLAVCEFP I

The amino acid sequence of the carbohydrate recognition domain (CRD) of human MBL (SEQ ID NO:4) is:

VGNKFFLING EIMTFEKVKA LCVKFQASVA TPRNAAENGA
IQNLIKEEAF LGITDEKTEG QFVDLTGNRL TYTNWNEGEP
NNAGSDEDCV LLLKNGQWND VPCSTSHLAV CEFPI

The amino acid sequence of the neck and carbohydrate recognition domain of MBL (SEQ ID NO:5) is:

PDGDSSLAAS ERKALQTEMA RIKKWLTFSL GKQVGNKFFL
TNGEIMTFEK VKALCVKFQA SVATPRNAAE NGAIQNLIKE
EAFLGITDEK TEGQFVDLTG NRLTYTNWNE GEPNNAGSDE
DCVLLLKNGQ WNDVPCSTSH LAVCEFPI

The truncated forms of the naturally-occurring protein include portions of any one of SEQ ID NOs:1-5 lacking 5, 10, 15, 20, 25, 30, 35, 40, 45, 50, 55, 60, 65, 70, 75, 80 or more amino acids from the amino-terminus of the protein, or the carboxy-terminus of the protein, or internally within the protein, or any combination thereof.

Alternatively, the truncated forms of the naturally-occurring protein of any one of SEQ ID NOs:1-5 have a deletion of at least 1%, 2%, 3%, 4%, 5%, 6%, 7%, 8%, 9%, 10%, 15%, 20%, 25%, 30%, 35%, 40%, 45% or 50% of the amino acids from the amino-terminus of the protein, or the carboxy-terminus of the protein, or internally within the protein, or any combination thereof.

As to particularly useful truncated forms of the protein, one example is the full-length amino acid sequence of the carbohydrate recognition domain (CRD) of MBL, shown in SEQ ID NO:4. In addition, suitable CRDs for use as the MBL of the invention include CRDs having an amino acid sequence of about 10 to about 110 amino acid residues, or about 50 to about 100 amino acid residues, of SEQ ID NO:4. In some aspects, the microbe surface-binding domain can have an amino acid sequence of at least about 5, at least about 10, at least about 15, at least about 20, at least about 30, at least about 40, at least about 50, at least about 60, at least about 70, at least about 80, at least about 90, at least about 100, at least about 110 amino acid residues or more, of SEQ ID NO:4. Accordingly, in some aspects, the carbohydrate recognition domain of an engineered MBL protein can comprise SEQ ID NO:4. In some aspects, the carbohydrate recognition domain of an engineered MBL protein can comprise a fragment of SEQ ID NO:4 as defined above. Also, exemplary amino acid sequences of such fragments include, but are not limited to, ND, EZN (where Z is any amino acid, e.g., P), NEGEPNNAGS (SEQ ID NO:10) or a fragment thereof comprising EPN, GSDEDCVLL or a fragment thereof comprising E, and LLLKNGQWNDVPCST (SEQ ID NO: 11) or a fragment thereof comprising ND. Modifications to such CRD fragments, e.g., by conservative substitution (i.e., where an amino acid is replace by an amino acid within the same class of amino acids, where the classes are: aliphatic amino acids (G, A, L, V, I); hydroxyl or sulfur/selenium-containing amino acids (S, C, U, T, M); aromatic amino acids (F, Y, W); basic amino acids (H, K, R); and acidic amino acids (D, E, N, Q)), are also within the scope described herein. In some aspects, the MBL or a fragment thereof used in the microbe surface-binding domain of the engineered MBLs described herein can be a wild-type molecule or a recombinant molecule.

The sequence variants of the naturally-occurring protein and the truncated forms thereof (e.g. SEQ ID NOs:1-5) include proteins having at least 50%, 55%, 60%, 65%, 70%, 75%, 80%, 85%, 90%, 91%, 92%, 93%, 94%, 95%, 96%, 97%, 98% or 99% sequence identity to any one of SEQ ID NOs:1-5, or truncated forms thereof, that retain the ability to reduce platelet activation in blood of the protein upon which they are based.

### FcMBL

In some aspects and embodiments of the invention, MBL is fusion protein comprising MBL, as defined above, and a second protein. An exemplary fusion protein comprises some or all of naturally-occurring MBL, such as the carbohydrate recognition domain (CRD) of MBL, and a portion of an immunoglobulin, such as the Fc domain. In use, the Fc domain dimerizes and strengthens the avidity and affinity of the binding by MBLs to monomeric sugars. In some aspects, the N-terminus of fusion proteins can further comprise an oligopeptide linker adapted to bind a solid substrate and orient the CRD of the MBL domain away from a substrate to which it is immobilized. As discussed above, engineered forms of MBLs are known in the art and include each of the forms of MBL disclosed in U.S. Pat. No. 9,150,631, U.S. Patent Pub. 2016/0311877, U.S. Patent Pub. 2019/0077850, U.S. Pat. Nos. 9,593,160, 10,435,457, U.S. Patent Pub. 2015/0173883 and International Application Publication No. WO 2011/090954, the entire disclosures of which are hereby incorporated by reference in their entirety.

FcMBL is a specific engineered form of MBL of the invention that comprises the neck and CRD domains of MBL linked to an IgG Fc domain. Proline 81 of mature MBL (SEQ ID NO:2) is a convenient N-terminal point at which to begin the sequence of this engineered construct. For example, the neck and CRD domains (SEQ ID NO:5) of MBL are fused downstream (C-terminal) to the Fc domain of human IgG (Fc7). The Fc domain may include the CH2-CH3 interface of the IgG Fc domain, which contains the binding sites for a number of Fc receptors including Staphylococcal protein A. In use, the Fc domain dimerizes and strengthens the avidity and affinity of the binding by MBLs to monomeric sugars. FcMBL is described in detail in U.S. Pat. No. 9,150,631, the entire disclosure of which is hereby incorporated by reference in its entirety.

Specific examples of FcMBLs that may be used in each of the aspects and embodiments of the invention include, but are not limited to, proteins where the neck and CRD domains of MBL are linked to an Fc component of human IgG1, with examples of the resulting constructs set forth in SEQ ID NOs:6, 7 and 9, and proteins where the CRD domain alone of MBL is linked to an Fc component of human IgG1, with an example of the resulting construct set forth in SEQ ID NO:8.

FcMBL.81 (SEQ ID NO: 6):
EPKSSDKTHT CPPCPAPELL GGPSVFLFPP KPKDTLMISR
TPEVTCVVVD VSHEDPEVKF NWYVDGVEVH NAKTKPREEQ
YNSTYRVVSV LTVLHQDWLN GKEYKCKVSN KALPAPIEKT
ISKAKGQPRE PQVYTLPPSR DELTKNQVSL TCLVKGFYPS
DIAVEWESNG QPENNYKTTP PVLDSDGSFF LYSKLTVDKS
RWQQGNVFSC SVMHEALHNH YTQKSLSLSP GAPDGDSSLA
ASERKALQTE MARIKKWLTF SLGKQVGNKF FLTNGEINIT
FEKVKALCVK FQASVATPRN AAENGAIQNL IKEEAFLGIT
DEKTEGQFVD LTGNRLTYTN WNEGEPNNAG SDEDCVLLLK
NGQWNDVPCS TSHLAVCEFP I
AKT-FcMBL (SEQ ID NO: 7):
AKTEPKSSDK THTCPPCPAP ELLGGPSVFL FPPKPKDTLM
ISRTPEVTCV VVDVSHEDPE VKENWYVDGV EVHNAKTKPR
EEQYNSTYRV VSVLTVLHQD WLNGKEYKCK VSNKALPAPI
EKTISKAKGQ PREPQVYTLP PSRDELTKNQ VSLTCLVKGF
YPSDIAVEWE SNGQPENNYK TTPPVLDSDG SFFLYSKLTV
DKSRWQQGNV FSCSVMHEAL HNHYTQKSLS LSPGAPDGDS
SLAASERKAL QTEMARIKKW LTFSLGKQVG NKFFLINGEI
MTFEKVKALC VKFQASVATP RNAAENGAIQ NLIKEEAFLG
ITDEKTEGQF VDLTGNRLTY TNWNEGEPNN AGSDEDCVLL
LKNGQWNDVP CSTSHLAVCE FPI
FcMBL.111 (SEQ ID NO: 8):
EPKSSDKTHT CPPCPAPELL GGPSVFLFPP KPKDTLMISR
TPEVTCVVVD VSHEDPEVKF NWYVDGVEVH NAKTKPREEQ
YNSTYRVVSV LTVLHQDWLN GKEYKCKVSN KALPAPIEKT
ISKAKGQPRE PQVYTLPPSR DELTKNQVSL TCLVKGFYPS
DIAVEWESNG QPENNYKTTP PVLDSDGSFF LYSKLTVDKS
RWQQGNVFSC SVMHEALHNH YTQKSLSLSP GATSKQVGNK
FFLTNGEEVI TFEKVKALCV KFQASVATPR NAAENGAIQN
LIKEEAFLGI TDEKTEGQFV DLTGNRLTYT NWNEGEPNNA
GSDEDCVLLL KNGQWNDVPC STSHLAVCEF PI
FcMBL (SEQ ID NO: 9):
AKTEPKSSDK THTCPPCPAP ELLGGPSVFL FPPKPKDTLM
ISRTPEVTCV VVDVSHEDPE VKENWYVDGV EVHNAKTKPR
EEQYDSTYRV VSVLTVLHQD WLNGKEYKCK VSNKALPAPI
EKTISKAKGQ PREPQVYTLP PSRDELTKNQ VSLTCLVKGF
YPSDIAVEWE SNGQPENNYK TTPPVLDSDG SFFLYSKLTV
DKSRWQQGNV FSCSVMHEAL HNHYTQKSLS LSPGAPDGDS
SLAASERKAL QTEMARIKKW LIFSLGKQVG NKFFLTNGEI
MTFEKVKALC VKFQASVATP RNAAENGAIQ NLIKEEAFLG
ITDEKTEGQF VDLTGNRLTY TNWNEGEPNN AGSDEDCVLL
LKNGQWNDVP CSTSHLAVCE FPI

In SEQ ID NO:9, the residues with a single underscore correspond to the Fc portion, the residues with a double underscore correspond to the MBL neck, and those residues without underscore correspond to the MBL carbohydrate-binding domain.

Various genetically engineered versions of MBL (e.g., FcMBL) are described in International Application Pub. Nos. WO 2011/090954 and WO 2013/012924, as well as U.S. Pat. Nos. 9,150,631 and 9,593,160, the contents of each of which are incorporated herein by reference in their entireties. Lectins and other mannan binding molecules are also described in, for example, U.S. Pat. Nos. 9,150,631 and 9,632,085, and PCT application nos. PCT/US2011/021603, PCT/US12/047201 and PCT/US13/028409, the contents of all of which are incorporated herein by reference in their entireties.

In aspects where the MBL is FcMBL, the Fc region or a fragment thereof can comprise at least one mutation, e.g., to modify the performance of the engineered MBL. For example, in some aspects, the half-life of the engineered MBL described herein can be increased, e.g., by mutating the lysine (K) at the residue 232 to alanine (A) as shown in the Fc domain sequence provided in SEQ ID NO:12. Other mutations, e.g., located at the interface between the CH2 and CH3 domains shown in Hinton et al (2004) J Biol Chem. 279:6213-6216 and Vaccaro C. et al. (2005) Nat Biotechnol. 23: 1283-1288, can be also used to increase the half-life of the IgG1 and thus the engineered MBL.

SEQ ID NO: 12:
EPKSSDKTHT CPPCPAPELL GGPSVFLFPP KPKDTLMISR
TPEVTCVVVD VSHEDPEVKF NWYVDGVEVH NAKTKPREEQ
YNSTYRVVSV LTVLHQDWLN GKEYKCKVSN KALPAPIEKT
ISKAKGQPRE PQVYTLPPSR DELTKNQVSL TCLVKGFYPS
DIAVEWESNG QPENNYKTTP PVLDSDGSFF LYSKLTVDKS
RWQQGNVFSC SVMHEALHNH YTQKSLSLSP GA

In some aspects and embodiments, the FcMBL of the invention comprises or consists of an amino acid sequence having at least 50%, 55%, 60%, 65%, 70%, 75%, 80%, 85%, 90%, 91%, 92%, 93%, 94%, 95%, 96%, 97%, 98% or 99% sequence identity to any one of SEQ ID NOs:6-9, that retain the ability to reduce platelet activation in blood of the protein upon which they are based.

The exemplary MBL sequences provided herein are not construed to be limiting. For example, while the exemplary sequences provided herein are derived from a human species, amino acid sequences of the same carbohydrate recognition domain in plants and other animal species such as mice, rats, porcine, bovine, feline, and canine are known in the art and within the scope described herein.

### Collectins

In addition to the aspects and embodiments of the invention defined above that comprise the collectin MBL (whether naturally-occurring or engineered), the present invention encompasses use of any other collectin that reduces the amount of platelet activation in the blood, when used in conjunction with the methods and components of the invention (e.g., coatings, films and substrates). Thus, any of the following additional collectins may also be used in the aspects and embodiments of the invention as defined herein: (i) surfactant protein A (SP-A; SEQ ID NO:13), (ii) surfactant protein D (SP-D; SEQ ID NO:14), (iii) collectin liver 1 (CL-Li; SEQ ID NO: 15), (iv) collectin placenta 1 (CL-P1; SEQ ID NO:16), and (v) collectin kidney 1 (CL-K1; SEQ ID NO:17.

Surfactant protein A (SP-A; Homo sapiens; GenBank: AAA36632.1; SEQ ID NO:13):

MTFKNNTTLS LERLFNSFPL HKGYITTREL HQLALANLIF
MENCADLHFP PQMRKCFPIP GTSCLFCPKT KTLFPVLHLA
NQYLCFLCQH KHCFPQTRFP DSQPSQATSR CVLSIKFTSS
IYINSHNYTL ILTCIIVRVL CPPVGCKSYK DRGHIHLAHP
SVPSTCHGSY QLTSTQYIVW ISKYDSNFQH PKLVYSPTEP
AQVEHMQCFL CMCLQREERE ALLLLPRVTI LTRLSAESTD
ERDGDSEPVN AVCRTALAFV PHESNVMLGI HNLLIWLL

Surfactant protein D (SP-D; Homo sapiens; GenBank: AAB59450.1; SEQ ID NO:14):

MLLFLLSALV LLTQPLGYLE AEMKTYSHRT MPSACTLVMC
SSVESGLPGR DGRDGREGPR GEKGDPGLPG AAGQAGMPGQ
AGPVGPKGDN GSVGEPGPKG DTGPSGPPGP PGVPGPAGRE
GALGKQGNIG PQGKPGPKGE AGPKGEVGAP GMQGSAGARG
LAGPKGERGV PGERGVPGNT GAAGSAGAMG PQGSPGARGP
PGLKGDKGIP GDKGAKGESG LPDVASLRQQ VEALQGQVQH
LQAAFSQYKK VELFPNGQSV GEKIFKTAGF VKPFTEAQLL
CTQAGGQLAS PRSAAENAAL QQLVVAKNEA AFLSMTDSKT
EGKFTYPTGE SLVYSNWAPG EPNDDGGSED CVEIFTNGKW
NDRACGEKRL VVCEF

Collectin liver 1 (CL-Li; also known as collectin-10; Homo sapiens; NCBI Reference Sequence: NP_006429.2; SEQ ID NO:15):

MNGFASLLRR NQFILLVLFL LQIQSLGLDI DSRPTAEVCA
THTISPGPKG DDGEKGDPGE EGKHGKVGRM GPKGIKGELG
DMGDQGNIGK TGPIGKKGDK GEKGLLGIPG EKGKAGTVCD
CGRYRKFVGQ LDISIARLKT SMKFVKNVIA GIRETEEKFY
YIVQEEKNYR ESLTHCRIRG GMLAMPKDEA ANTLIADYVA
KSGFFRVFIG VNDLEREGQY MFTDNTPLQN YSNWNEGEPS
DPYGHEDCVE MLSSGRWNDT ECHLTMYFVC EFIKKKK

Collectin placenta 1 (CL-P1; Homo sapiens; GenBank: BAB72147.1; SEQ ID NO:16):

MKDDFAEEEE VQSFGYKRFG IQEGTQCTKC KNNWALKESI
ILLYILCALL TITVAILGYK VVEKMDNVTG GMETSRQTYD
DKLTAVESDL KKLGDQTGKK AISTNSELST FRSDILDLRQ
QLREITEKTS KNKDTLEKLQ ASGDALVDRQ SQLKETLENN
SFLITTVNKT LQAYNGYVTN LQQDTSVLQG NLQNQMYSHN
VVIMNLNNLN LTQVQQRNLI TNLQRSVDDT SQAIQRIKND
FQNLQQVFLQ AKKDTDWLKE KVQSLQTLAA NNSALAKANN
DTLEDMNSQL NSFTGQMENI TTISQANEQN LKDLQDLHKD
AENRTAIKFN QLEERFQLFE TDIVNIISNI SYTAHHLRTL
TSNLNEVRTT CTDTLTKHTD DLTSLNNTLA NIRLDSVSLR
MQQDLMRSRL DTEVANLSVI MEEMKLVDSK HGQLIKNFTI
LQGPPGPRGP RGDRGSQGPP GPTGNKGQKG EKGEPGPPGP
AGERGPIGPA GPPGERGGKG SKGSQGPKGS RGSPGKPGPQ
GPSGDPGPPG PPGKEGLPGP QGPPGFQGLQ GTVGEPGVPG
PRGLPGLPGV PGMPGPKGPP GPPGPSGAVV PLALQNEPTP
APEDNGCPPH WKNFTDKCYY FSVEKEIFED AKLFCEDKSS
HLVFINTREE QQWIKKQMVG RESHWIGLTD SERENEWKWL
DGTSPDYKNW KAGQPDNWGH GHGPGEDCAG LIYAGQWNDF
QCEDVNNFIC EKDRETVLSS AL

Collectin kidney 1 (CL-K1; also known as collectin-11; Homo sapiens; GenBank: BAF43301.1; SEQ ID NO:17):

MRGNLALVGV LISLAFLSLL PSGHPQPAGD DACSVQILVP
GLKGDAGEKG DKGAPGRPGR VGPTGEKGDM GDKGQKGSVG
RHGKIGPIGS KGEKGDSGDI GPPGPNGEPG LPCECSQLRK
AIGEMDNQVS QLTSELKFIK NAVAGVRETE SKIYLLVKEE
KRYADAQLSC QGRGGTLSMP KDEAANGLMA AYLAQAGLAR
VFIGINDLEK EGAFVYSDHS PMRTFNKWRS GEPNNAYDEE
DCVEMVASGG WNDVACHTTM YFMCEFDKEN M

As with MBL, both naturally-occurring collectins and engineered forms of the proteins may be used in the invention. Engineered forms of the proteins include, but are not limited to, truncated forms of the naturally-occurring proteins, sequence variants of the naturally-occurring proteins, sequence variants of the truncated forms of the proteins, fusion proteins comprising the naturally-occurring protein, fusion proteins comprising the truncated forms of the proteins, and fusion proteins comprising the sequence variants.

The truncated forms of the naturally-occurring collectins include portions of any one of SEQ ID NOs:13-17 lacking 5, 10, 15, 20, 25, 30, 35, 40, 45, 50, 55, 60, 65, 70, 75, 80 or more amino acids from the amino-terminus of the protein, or the carboxy-terminus of the protein, or internally within the protein, or any combination thereof.

Alternatively, the truncated forms of the naturally-occurring protein of any one of SEQ ID NOs:13-17 have a deletion of at least 1%, 2%, 3%, 4%, 5%, 6%, 7%, 8%, 9%, 10%, 15%, 20%, 25%, 30%, 35%, 40%, 45% or 50% of the amino acids from the amino-terminus of the protein, or the carboxy-terminus of the protein, or internally within the protein, or any combination thereof.

The sequence variants of the naturally-occurring protein and the truncated forms thereof (e.g. SEQ ID NOs:13-17) include proteins having at least 50%, 55%, 60%, 65%, 70%, 75%, 80%, 85%, 90%, 91%, 92%, 93%, 94%, 95%, 96%, 97%, 98% or 99% sequence identity to any one of SEQ ID NOs:13-17, or truncated forms thereof, that retain the ability to reduce platelet activation in blood of the protein upon which they are based.

Each of the collectins of the invention (e.g., MBL) may include one or more detectable labels indicative of binding to the CRD domain by target microbes and/microbial matter (e.g., MAMPs). Detectable labels can produce a detectable signal indicative of the presence of a target and which are detectable by spectroscopic, photochemical, biochemical, immunochemical, electrical, optical or chemical means. Suitable labels include biotin, fluorescent molecules, radioisotopes, nucleotide chromophores, enzymes, substrates, chemiluminescent moieties, bioluminescent moieties, and the like.

### Films and Coatings

The invention includes films and coatings comprising collectins (e.g., MBL), as defined herein. Such films and coatings include hydrogels, hydrophilic polymers, phosphorylcholine polymer coatings, albumin-heparin coatings, to name only a few, that are applied to surfaces of more durable biomaterials (e.g. polyurethane catheters) in an effort to decrease their thrombogenicity.

### Coated Substrates

The invention includes substrates coated with one or more species of collectins (e.g., MBL), as defined herein. In some aspects, the substrate is a solid substrate. Examples of solid substrate include, but are not limited to, beads or particles (including nanoparticles, microparticles, polymer microbeads, magnetic microbeads, and the like), filters, fibers, screens, mesh, membranes, semi-permeable membranes, permeable membranes, cartridges, tubes, hollow fibers, scaffolds (such as cell-culture scaffolds), plates (such as cell-culture plates), channels, gold particles, magnetic materials, medical apparatuses (e.g., needles, catheters and syringes), appliances (such as a medical or dental appliance), implants (such as a medical or dental implant), medical components, dialyzers, hemodialyzers, dipsticks or test strips, filtration devices or membranes, hollow fiber cartridges, microfluidic devices, mixing elements (e.g., spiral mixers), and components of devices, including medical devices and therapeutic devices, such as filtration devices, oxygenation devices, extracorporeal devices (e.g. ECMO devices), dialysis devices, infusion devices, drainage devices, pumps and other substrates commonly utilized in assay formats, and any combinations thereof.

The collectins may be coated and/or immobilized onto the substrate such that it coats all surfaces of the substrate, or only selected portions of the substrate.

Immobilization (via coating) of collectins onto the substrate can be either non-specific (e.g., by adsorption to the surface) or specific (e.g. where another molecule, such as a linker exhibiting binding specificity for the collectin, immobilized on the substrate is used to capture the collectin). Collectins may be linked to the substrate surface through one or more linkers which may be cleavable to accommodate release or elution of the bound target molecules for subsequent analysis. Collectins may attach to the one or more substrates though a covalent linking process. Substrate linkage may be accomplished through, for example, biotin-avidin binding, 3-(3-dimethylaminopropyl)carbodiimide (EDC), 1-Ethyl-3-[3-dimethylaminopropyl]carbodiimide hydrochloride, hydroxybenzotriazole (HOBT), N-Hydroxysuccinimide (NHS), 2-(1H-7-Azabenzotriazol-1-yl)-1,1,3,3-tetramethyl uronium hexafluorophosphate methanaminium (HATU), silanization, surface activation through plasma treatment, and the like.

In some embodiments, the substrate is fabricated or coated with a material prior to being coated by collectins, where the material is one or more of polydimethylsiloxane, polyimide, polyethylene terephthalate, polymethylmethacrylate, polyurethane, polyvinylchloride, polystyrene polysulfone, polycarbonate, polymethylpentene, polypropylene, a polyvinylidine fluoride, polysilicon, polytetrafluoroethylene, polysulfone, acrylonitrile butadiene styrene, polyacrylonitrile, polybutadiene, poly(butylene terephthalate), poly(ether sulfone), poly(ether ether ketones), poly(ethylene glycol), styrene-acrylonitrile resin, poly(trimethylene terephthalate), polyvinyl butyral, polyvinylidenedifluoride, poly(vinyl pyrrolidone), and any combination thereof.

In some embodiments, collectins can be conjugated to the substrate by methods well known in the art for conjugating peptides with other molecules. For example, Hermanson, BIOCONJUGATE TECHNIQUES (2nd Ed., Academic Press (2008)) and Niemeyr, Bioconjugation Protocols: Strategies & Methods, in METHODS IN MOLECULAR BIOLOGY (Humana Press, 2004), provide a number of methods and techniques for conjugating peptides to other molecules. de Graaf, et al., 20 Biocojugate Chem. 1281 (2009), provides a review of site-specific introduction of non-natural amino acids into peptides for conjugation.

Alternatively, the surface of the substrate can be functionalized to include binding molecules that bind selectively with the collectins. The binding molecule can be bound covalently or non-covalently on the surface of the substrate. As used herein, the term “binding molecule” refers to any molecule that is capable of specifically binding a collectin, as defined herein, and thus links the collectin to the surface, such that it is displayed on the surface. Representative examples of binding molecule include, but are not limited to, antibodies, antigens, lectins, proteins, peptides, nucleic acids (DNA, RNA, PNA and nucleic acids that are mixtures thereof or that include nucleotide derivatives or analogs); receptor molecules, such as the insulin receptor; ligands for receptors (e.g., insulin for the insulin receptor); and biological, chemical or other molecules that have affinity for another molecule, such as biotin and avidin. The binding molecules need not comprise an entire naturally occurring molecule but may consist of only a portion, fragment or subunit of a naturally or non-naturally occurring molecule, as for example the Fab fragment of an antibody. The binding molecule may further comprise a marker that can be detected.

The binding molecule can be conjugated to surface of the substrate using any of a variety of methods known to those of skill in the art. The binding molecule can be coupled or conjugated to surface of the substrate covalently or non-covalently. Covalent immobilization may be accomplished through, for example, silane coupling. See, e.g., Weetall, 15 Adv. Mol. Cell Bio. 161 (2008); Weetall, 44 Meths. Enzymol. 134 (1976). The covalent linkage between the binding molecule and the surface can also be mediated by a linker. The non-covalent linkage between the binding molecule and the surface can be based on ionic interactions, van der Waals interactions, dipole-dipole interactions, hydrogen bonds, electrostatic interactions, and/or shape recognition interactions.

As used herein, the term “linker” means a molecular moiety that connects two parts of a composition. Peptide linkers may affect folding of a given fusion protein, and may also react/bind with other proteins, and these properties can be screened for by known techniques. Example linkers, in addition to those described herein, include is a string of histidine residues, e.g., His6; sequences made up of Ala and Pro, varying the number of Ala-Pro pairs to modulate the flexibility of the linker; and sequences made up of charged amino acid residues e.g., mixing Glu and Lys. Flexibility can be controlled by the types and numbers of residues in the linker. See, e.g., Perham, 30 Biochem. 8501 (1991); Wriggers et al., 80 Biopolymers 736 (2005). Chemical linkers may comprise a direct bond or an atom such as oxygen or sulfur, a unit such as NH, C(O), C(O)NH, SO, SO₂, SO₂NH, or a chain of atoms, such as substituted or unsubstituted C₁-C₆ alkyl, substituted or unsubstituted C₂-C₆ alkenyl, substituted or unsubstituted C₂-C₆ alkynyl, substituted or unsubstituted C₆-C₁₂ aryl, substituted or unsubstituted C₅-C₁₂ heteroaryl, substituted or unsubstituted C₅-C₁₂ heterocyclyl, substituted or unsubstituted C₃-C₁₂ cycloalkyl, where one or more methylenes can be interrupted or terminated by O, S, S(O), SO₂, NH, or C(O).

In some aspects, the substrate is a hemodialyzer. Some or all of the components comprising the hemodialyzer, such as a semi-permeable membrane, may be coated with collectins.

In some aspects, the substrate is a filter, such as filer paper, e.g., cellulose, or a membrane filter, such as regenerated cellulose, cellulose acetate, nylon, PTFE, polypropylene, polyester, polyethersulfone, polycarbonate, and polyvinylpyrolidone. The filter may be coated with collectins.

In some aspects, the substrate is a component of a therapy device. In some aspects, the therapy device can comprise a coating on any of one or more internal components of the therapy device that results in the immobilization of collectins on the one or more components. For example, where the therapy device is an oxygenation device comprising a membrane, the collectins can be coated on the membrane.

In some aspects, the substrate is a collectin-coated component of an oxygenation device. Examples of an oxygenation device are described in U.S. Application Pub. No. 2019/0167882, which is incorporated by reference in its entirety herein.

In some aspects, the substrate is a collectin-coated component of an ECMO device. Examples of an ECMO device are described in U.S. Application Pub. No. 2019/0167882, which is incorporated by reference in its entirety herein.

In some aspects, the substrate is a collectin-coated component of a dialysis device, for example a hemodialysis device or a peritoneal dialysis device.

In some aspects, the substrate is a collectin-coated component of an infusion device or a transfusion device. For example, the fluid can be sourced from a patient, a different patient or a fluid storage device, and the fluid deposit can be the patient. In some aspects, the infusion device can comprise a syringe comprising a filtering device, where a fluid from a subject such as blood can be drawn into the syringe, filtered, and returned to the subject, and where the syringe is coated with collectins.

In some aspects, the substrate is a collectin-coated component of a drainage device. In these aspects, the fluid can be a bodily fluid, the fluid source can be a patient, and the fluid deposit can be the patient, a different patient or a fluid storage system. For example, a patient with hydrocephalus may require drainage of cerebrospinal fluid using a therapy device such as a percutaneous drainage device, and a filtration device can be coupled to the drainage line to filter the fluid. The fluid can then be analyzed for diagnostic applications, as described herein. In some aspects, the percutaneous drainage device can be used to drain a biliary, pleural or an abscess.

In some aspects, the system comprises a collectin-coated component of a pump, where the pump can comprise a negative or positive pressure pump depending on the system configuration. In some aspects, the fluid source is a higher pressure than the fluid deposit, and a pump is optional. For example, a fluid source can be the artery or vein of a patient, and the fluid deposit can be the vein of the patient.

In addition to coating substrates with collectins to reduce unintended platelet activation and coagulation, to reduce bleeding, to reduce antibiotic depletion and/or to reduce ficolin-2 depletion, substrates may be coated with anticoagulants such as heparin, warfarin, rivaroxaban, dabigatran, apixaban, edoxaban, enoxaparin, or fondaparinux to further reduce the risk of thrombosis. In some aspects, the surface of the materials can be modified to reduce coagulation. Examples of coatings for surface modification include but are not limited to: Poly(ethylene oxide) (PEO) to increase the surface hydrophilicity; Albumin to reduce platelet adhesion; Pyrolytic carbon to reduce platelet adhesion and spreading on the surface; Phosphorylcholine surfaces that are predominantly lipid having a physiologically neutral pH on the outer surface of non-activated cell membranes to reduce protein and cell adhesion; Elastin-inspired polymer or synthesized elastin-inspired polymers to decreased fibrinogen adsorption and reduce proinflammatory cytokine release from monocytes; CTI to inhibit the activation of fXII and attenuate the deposition of protein; Immobilized heparin or heparin-mimicking molecules to activate antithrombin and attenuate the inflammatory response; a direct thrombin inhibitor grafted surface such as hirudin, bivalirudin, or argatroban to inhibit thrombin; and Thrombomodulin or recombinant-thrombomodulin to promote the activation of protein C, thus limiting the coagulation by inactivating fVIIIa and fVa, important cofactors for fXa and thrombin generation.

### Methods

The invention includes systems and methods that make use of the collectin-coated substrates of the invention, as well as collectin-based coatings and films of the invention. Such systems and methods include, for example, methods for reducing platelet activation in blood, and methods for reducing platelet activation in a subject undergoing a medical procedure. Such systems and methods also include, for example, methods for processing fluids, such as blood, without depletion or loss of antibiotics and/or ficolin-2.

The invention provides systems and methods for treating fluids, such as biological fluids including blood, as the fluids flow through a system. Such systems and methods can be used in both therapeutic and diagnostic applications. As such, fluids such as blood or other fluids may be treated (e.g., oxygenated, removal of carbon dioxide, combined with an agent such as a drug) and filtered to remove harmful pathogens before reintroduction of the fluid into a subject (e.g., a human or an animal patient) and/or to capture microbes for further analysis (e.g., detection, identification and antimicrobial susceptibility testing). The inclusion of at least one collectin-coated substrate in the systems and methods of the invention result in a reduction in unintended platelet activation and coagulation in the fluids, such as blood, as it flows through the system. Alternatively, or in addition, inclusion of at least one collectin-coated substrate in the systems and methods of the invention results in fewer complications associated with bleeding that can result from depletion of platelets, if present in the fluid, such as blood, as it flows through the system. Alternatively, or in addition, inclusion of at least one collectin-coated substrate in the systems and methods of the invention results in less depletion of antibiotics, if present in the fluid, such as blood, as it flows through the system. Alternatively, or in addition, inclusion of at least one collectin-coated substrate in the systems and methods of the invention results in less depletion of ficolin-2, if present in the fluid, such as blood, as it flows through the system.

Microbes captured or filtered using systems and methods of the invention can include, for example, living or dead Gram-positive bacterial species, Gram-negative bacterial species, mycobacteria, fungi, parasites, viruses, or portions thereof. By combining these functions, a subject can receive a therapy, e.g., oxygenation of blood, with a reduced risk of unintended platelet activation (e.g. thrombosis), reduced risk of bleeding (due to platelet depletion), reduced risk of antibiotic depletion and/or reduced risk of ficolin-2 depletion, while simultaneously removing pathogens and components thereof such as pathogen associated molecular patterns (PAMPs) from the blood.

Systems and methods of the invention can include obtaining a fluid (e.g., a bodily fluid) from a fluid source such as a patient. In certain aspects, the fluid, such as blood, may be obtained directly from a patient, for example, from an artery or a vein of a patient, and connected to the system for treatment and filtration. In some aspects, the fluid source may be a vial or other container in which a fluid may be stored. The vial or other container, or the means used to obtain the fluid from the patient, e.g. a syringe, may be coated with collectins to reduce unintended platelet activation and coagulation, to reduce bleeding, to reduce antibiotic depletion and/or to reduce ficolin-2 depletion.

After treatment, the treated fluid may be deposited to a fluid deposit, for example, returned to the patient or a different patient, and/or the treated fluid may be retained for further processing, for storage, e.g., a blood bank, or discarded. In certain aspects, blood or other fluids are siphoned directly from the patient or other fluid source and the fluid's natural pressure or flow (e.g. the patient's blood pressure) is used to force the fluid through the system. For example, the fluid source can be an artery of a patient, and the fluid deposit can be a vein of a patient, where the patient can be the same or a different patient. One or more of the tubes, bags, systems and components used to move or transfer the fluids may be coated with collectins to reduce unintended platelet activation and coagulation, to reduce bleeding, to reduce antibiotic depletion and/or to reduce ficolin-2 depletion.

In some aspects, a pump may be used to drive the fluid through the system. Extracorporeal blood pumps including roller pumps, pulsatile tube compression pumps, ventricular pumps, and centrifugal pumps are known in the art and, among others are contemplated for use with the invention. One or more of the components of the pumps may be coated with collectins to reduce unintended platelet activation and coagulation, to reduce bleeding, to reduce antibiotic depletion and/or to reduce ficolin-2 depletion.

In each of these examples, it will be clear that at least one of the components to which the blood is exposed is coated with a collectin, such as MBL. In each of these examples, the use of a collectin-coated component reduces unintended platelet activation and coagulation, reduces bleeding, reduces antibiotic depletion and/or reduces ficolin-2 depletion.

In some aspects, microbes and/microbial matter (e.g., MAMPs) are captured by collectin-coated solid substrates. The invention includes methods of detecting the MAMPs. The MAMPs bound to collectin-coated (e.g., MBL-coated) solid supports or solid surfaces can be detected by any methods known in the art or as described herein. Examples of detection methods can include, but are not limited to, spectrometry, electrochemical detection, polynucleotide detection, fluorescence anisotropy, fluorescence resonance energy transfer, electron transfer, enzyme assay, magnetism, electrical conductivity, isoelectric focusing, chromatography, immunoprecipitation, immunoseparation, aptamer binding, filtration, electrophoresis, use of a CCD camera, immunoassay, ELISA, Gram staining, immunostaining, microscopy, immunofluorescence, western blot, polymerase chain reaction (PCR), RT-PCR, fluorescence in situ hybridization, sequencing, mass spectrometry, or substantially any combination thereof. The captured microbe can remain bound on the collectin-coated solid substrates during detection and/or analysis, or be isolated from the collectin-coated solid substrates prior to detection and/or analysis.

For example, after processing of blood, the captured MAMPs can be eluted through a variety of known methods to allow for subsequent analysis steps without interference of the collectin. Elution may be accomplished through any known means and will generally depend on the desired analysis method and the composition of the substrate and collectin. Exemplary elution methods include temperature-based (e.g., heating to 70° C. or more), physical (e.g., agitation), photosensitive cleavage, or chemical methods. In some aspects, elution through heating may be performed in calcium-free water. Exemplary chemical elution methods may involve a change in pH and/or application of a chelation agent. Chelation agents may include one or more of ethylenediaminetetraacetic acid (EDTA), calcium disodium edetate (CaNa2EDTA), ethylene glycol-bis(β-aminoethyl ether)-N,N,N′,N′-tetraacetic acid (EGTA), 1,2-bis(oaminophenoxy) ethane-N,N,N′,N′-tetraacetic acid (BAPTA), deferoxamine mesylate salt (DFOM).

After isolation of MAMPs from the fluid, the captured material can be digested for analysis. Digestion can refer to the release of constituent microbial components for subsequent analysis. Digestion can occur through exposure to a substance selected based on the desired analysis method and the target microbe to analyzed.

The present invention provides methods for treating a fluid, such a blood, wherein the amount of unintended platelet activation or coagulation is decreased, complications associated with bleeding are reduced, the amount of antibiotic depletion is decreased and/or the amount of ficolin-2 depletion is decreased. An example of such treatment is extracorporeal therapy. The method comprises providing a fluid from a fluid source to a system, treating the fluid in the system, and providing the fluid to a fluid deposit (e.g. back into the subject), wherein at least one component of the system to which the fluid is exposed comprises a collectin-coated substrate, as defined herein. In some aspects, treating the fluid in the system can include providing a therapy to the fluid using any of the therapy devices described herein and/or filtering the fluid using any of the filtering devices described herein. In some aspects, providing a therapy to a fluid comprises oxygenating the fluid and/or removing carbon dioxide from the fluid. In some aspects, providing a therapy to a fluid comprises adding an agent such as a drug to the fluid and/or infusing the fluid to a subject. In some aspects, providing a therapy to a fluid comprises removing, e.g., draining, the fluid from a subject. The method can optionally include analyzing the fluid, where analyzing the fluid comprises detecting and/or identifying one or more microbes or microbe components present in the fluid, for example using a mass spectrometric analysis method.

The present invention also provides a method for treating a subject such as a human or an animal comprising removing a fluid, such as blood, from a fluid source, providing the fluid to a system, treating the fluid in the system, and providing the fluid to a fluid deposit (e.g. back into the subject), wherein the amount of unintended platelet activation or coagulation is decreased, complications associated with bleeding are reduced, the amount of antibiotic depletion is decreased and/or the amount of ficolin-2 depletion is reduced. An example of such treatment is extracorporeal therapy. In some aspects, treating the fluid in the system can include providing a therapy to the fluid using any of the therapy devices described herein and/or filtering the fluid using any of the filtering devices described herein, wherein at least one component of the therapy device or filtering device comprises a collectin-coated substrate.

In some aspects, providing a therapy to a fluid comprises oxygenating the fluid and/or removing carbon dioxide from the fluid, such as during extracorporeal therapy. In some aspects, providing a therapy to a fluid comprises adding an agent such as a drug to the fluid and/or infusing the fluid to a subject. In some aspects, providing a therapy to a fluid comprises removing, e.g., draining, the fluid from a subject. The method can optionally include analyzing the fluid, where analyzing the fluid comprises detecting and/or identifying one or more microbes or microbe components present in the fluid, for example using a mass spectrometric analysis method.

An example includes treating a patient who is in need of oxygenated blood, where the method includes connecting a patient to a system where the system comprises a veno-venous oxygenation or ECMO device, and wherein at least one component of the veno-venous oxygenation or ECMO device comprises a collectin-coated substrate, as defined herein. The patient is connected to the system via cannulation where venous blood is siphoned into the oxygenation or ECMO device. Carbon dioxide is removed from the blood and/or blood is oxygenated and returned to the systemic venous circulation of the patient. Optionally, filtration device can be included in the system. Further optionally, a sample can be taken from the filtration device to identify any captured microbes or for antimicrobial susceptibility testing. Another exemplary method includes connecting a patient to a system where the system comprises an arterio-venous oxygenation or ECMO device, and wherein at least one component of the veno-venous oxygenation or ECMO device comprises a collectin-coated substrate, as defined herein. The patient is connected to the system via cannulation where arterial blood is siphoned into the oxygenation or ECMO device. Carbon dioxide is removed from the blood and/or blood is oxygenated and returned to the systemic venous circulation of the patient. Optionally, filtration device can be included in the system. Further optionally, a sample can be taken from the filtration device to identify the captured microbes or for antimicrobial susceptibility testing.

While it will be apparent that each of the inventions defined herein can be used in any patient population, it can be noted that the collectin-coated substrates and methods of the invention will be especially useful in patient populations that include, but not limited to, patients having a pathogenic infection (e.g. bacterial or viral infection, septicemia), patient being treated with antibiotics (e.g. patients receiving large doses of i.v. antibiotics), patients having collectin deficiency (i.e. collectin-deficient patients), patients having mannose-binding lectin deficiency (i.e. MBL-deficient patients), patients having a ficolin-2 deficiency (i.e. ficolin-2-deficient patients), and patients having cancer that are receiving chemotherapy or taking drugs that suppress the immune system. In such patients, collectin-coated substrates, such as components of medical devices, will be considered the “substrates of choice.”

In some aspects, compositions (e.g., engineered microbe targeting molecules as described further therein), methods, systems, and assays are further described in at least one of the following: U.S. Provisional Applications 61/296,222, 61/508,957, 61/604,878, 61/605,052, 61/605,081, 61/788,570, 61/846,438, 61/866,843, 61/917,705, 62/201,745, 62/336,940, 62/543,614; PCT application numbers PCT/US2011/021603, PCT/US2012/047201, PCT/US2013/028409, PCT/US2014/028683, PCT/US2014/046716, PCT/US2014/071293, PCT/US2016/045509, PCT/US2017/032928; U.S. patent application Ser. Nos. 13/574,191, 14/233,553, 14/382,043, 14/766,575, 14/831,480, 14/904,583, 15/105,298, 15/415,352, 15/483,216, 15/668,794, 15/750,788, 15/839,352, 16/059,799, 16/302,023, 16/553,635; and U.S. Pat. Nos. 9,150,631, 9,593,160, 9,632,085, 9,791,440, and 10,435,457; the contents of each of which are incorporated by reference herein in their entireties.

### Example 1

Commercially-available hemodialysis filters were coated with FcMBL (SEQ ID NO:9) in order to determine whether there would be a reduction in platelet and coagulation activation in blood exposed to such filters, versus filters lacking the collectin.

FcMBL was covalently attached via an amide bond to the inner lumen of the hollow fibers that make up Baxter Revaclear™ 400 hemofilters. The resulting filters (termed Filtriva™ devices herein) were compared to control Revaclear™ 400 filters that lacked FcMBL coating.

FcMBL of SEQ ID NO:9 is a 383-amino acid fully human recombinant fusion protein expressed in CHO cells that comprises the carbohydrate-binding and neck domains of MBL for pathogen capture and the Fc domain of human immunoglobulin IgG1. The MW of the FcMBL monomer is 42,583 Da, and the protein exists primarily as a hexamer under non-reducing conditions. FcMBL contains no known glycosylation or other post-translational modifications.

For these experiments, FcMBL was purified from cell cultures prepared to produce the protein. Only animal component-free and chemically defined reagents were used to generate the FcMBL-expressing cell lines. Purification included filtration, Protein A chromatography, anion exchange chromatography, and ultrafiltration/diafiltration. The purified protein was tested for purity, potency and safety, among other attributes. The purified protein was maintained as a frozen solution at a protein concentration of ˜2 mg/mL prior to use in a neutral phosphate buffered saline solution comprised of 8 mM Na₂HPO₄, 4 mM KH₂PO₄, 2.7 mM KCl, 137 mM NaCl, pH 7.0.

### Preparing Filtriva™ Dialyzer

The Filtriva™ devices were prepared using a commercially available hollow-fiber hemodialyzer, the Revaclear™ 400 capillary dialyzer manufactured by Baxter, Inc. (K130039).

The Revaclear™ 400 hemodialyzer was removed from its original sterile packaging and dried to eliminate residual moisture with filtered, compressed air. The device was next treated with a carbon dioxide plasma to introduce carboxylate moieties to enable the chemical crosslinking of the FcMBL protein in the subsequent processing step.

The hollow fibers of the hemodialyzer were filled with a 2-(N-morpholino) ethanesulfonic acid (MES) buffer (0.1M MES, pH 5.0) containing 25 mg of FcMBL and 1-ethyl-3-(3-dimethylaminopropyl)carbodiimide (EDC), a zero-length carboxyl-to-amino crosslinker. While filling the hollow fibers with the MES/EDC/FcMBL solution (EDC:FcMBL ratio of 1000:1), the dialysate compartment was filled with 0.1M MES, pH 5.0 buffer only (i.e. no FcMBL; no EDC). After the devices were filled, they were capped and incubated overnight at 2-8° C. during which time the EDC crosslinked the FcMBL protein to the inner lumen of the hollow fibers.

The following day, the FcMBL-coupled devices were washed with excess phosphate buffered saline (PBS), pH 7.4 containing 10 mM ethylenediaminetetraacetic acid (EDTA) (PBS/EDTA) through both the blood and dialysate compartments to remove the MES buffer, unbound FcMBL, and EDC. In flow and outflow samples were obtained throughout the process to verify FcMBL protein coupling. The PBS/EDTA-filled Filtriva™ devices were capped, the caps were sealed with a heat-shrink wrapper, the devices were double-bagged, and then stored at 2-8° C. until use.

### Determination of FcMBL Bound to the Dialyzer Membrane

The amount of FcMBL protein chemically crosslinked to the inner lumen of the plasma treated hollow fibers was computed via a mass balance that considered the mass of protein that was input into the Revaclear™ 400 in the EDC-FcMBL-MES solution (Input) and the mass of protein that flowed out of the blood compartment of the device (Blood Port Output) after the incubation period. The difference in protein amounts between Input and Blood Port Output samples was assumed to be bound to the membrane.

The FcMBL protein concentration in each sample was determined using the Pierce 660 nm assay and an FcMBL standard curve.

Table 1 contains a summary of the mean and standard deviation of the protein amount that was introduced into the blood compartment (Inflow) and flowed out of the blood compartment after incubation (Outflow) for 12 development lots of Filtriva™. The amount of FcMBL bound to the membrane was computed from the difference between Inflow and Outflow. As demonstrated in Table 1, the average Filtriva™ device contained 15.4 mg of coupled FcMBL (mean across n=180 total devices). The standard deviation across the 180 devices was approximately 2 mg of FcMBL.

TABLE 1
FcMBL protein coupling across 12 development lots of
Filtrivas ™ (n = 180 total devices)

| | Inflow (mg) | | Bound (mg) | | Outflow (mg) | |
|---|---|---|---|---|---|---|
| | Mean | Stdev | Mean | Stdev | Mean | Stdev |
| B-152-03072017 | 26.01 | 0.34 | 14.57 | 1.44 | 11.43 | 1.46 |
| B-152-03132017 | 25.89 | 1.19 | 16.03 | 3.25 | 9.86 | 2.64 |
| B-152-03152017 | 25.43 | 0.60 | 14.92 | 1.76 | 10.51 | 1.74 |
| B-152-03202017 | 24.26 | 0.63 | 13.08 | 1.30 | 11.19 | 1.07 |
| B-152-03222017 | 22.77 | 2.38 | 14.39 | 1.93 | 8.38 | 1.40 |
| B-152-03272017 | 24.63 | 0.44 | 13.56 | 0.98 | 11.07 | 0.96 |
| B-152-03292017 | 24.13 | 0.48 | 15.58 | 0.96 | 8.54 | 0.84 |
| B-152-04032017 | 25.85 | 0.40 | 15.11 | 1.02 | 10.73 | 0.95 |
| B-152-04052017 | 25.54 | 0.63 | 13.15 | 1.77 | 12.40 | 1.65 |
| B-152-05102017 | 30.61 | 0.70 | 16.00 | 1.35 | 14.61 | 1.01 |
| B-152-05312017 | 31.87 | 0.55 | 18.89 | 1.27 | 12.98 | 1.21 |
| B-152-06212017 | 32.41 | 0.45 | 19.25 | 1.42 | 13.16 | 1.22 |
| Average = | 26.62 | | 15.38 | | 11.24 | |
| Stdev = | 3.18 | | 1.99 | | 1.85 | |

Three Filtrivas from Lot 152-04052017 (Devices #1, #2, and #3) were selected for analysis of their respective PBS/EDTA wash fractions to determine the extent to which the FcMBL bound to the membrane, remains bound under flow conditions. The mass of protein obtained from five serial fractions of 45 mL, three fractions from the Blood Port Output and two fractions from the subsequent Wash Buffer, was determined. The protein that was measured in Blood Port Output 1 and 2 (total volume ˜90 mL, consistent with the total blood compartment volume of 93 mL) was the residual protein from the EDC-FcMBL-MES solution that did not covalently couple to the membrane during incubation (data not shown). After the second Blood Port Output, the amount of FcMBL in the subsequent fractions was undetectable (data not shown). From these data it was concluded that the FcMBL covalently coupled to the membrane stays bound under flow conditions and is not released upon subsequent washing/flushing with PBS/EDTA.

### Platelet Activation Testing

Experiments were performed with freshly donated heparinized human blood (5.0 IU/mL). Blood from one donor (˜500 mL) was divided and used for one Filtriva™ and one Revaclear™ 400, respectively (240 mL per filter). This setup was repeated three times, leading to three independent experiments (n=3) in which each pair of filters was compared with the same donor blood. Blood and dialysate compartment was primed separately starting with the blood compartment by recirculating 1 L of saline at 250 mL/min for 30 min, followed by flushing 1 L saline at 60 mL/min in single pass, respectively. Saline was replaced by human blood (37° C., 200 mL/min). The volume of saline was discarded and the blood flow-rate was adjusted to QB=400 mL/min (defined as t=0 min). Therefore, at t=0 min, the blood has contacted each filter for a short period of time. The dialysate ports remained clamped during the whole experiment. Samples were taken from the arterial port of the filter at the start and at time points 12, 30, 120, 180 and 240 min for the measurement of number of WBC, number of RBC, platelet count (PC), heparin concentration, release of platelet factor 4 (PF4), generation of the thrombin-antithrombin complex III (TAT) and for flow-cytometric analysis of platelet activation (CD41a, CD62P).

TABLE 2

| | | | | Surface | Steril- |
|---|---|---|---|---|---|
| | | | Exp. | area | ization |
| Filter | Lot | Device Number | date | (m²) | mode |
| Filtriva | 329740 | B17060053-012 | July | 1.8 | gamma |
| | | B17060053-013 | 2018 | | |
| | | B17060053-014 | | | |
| Revaclear 400 | 7-7013- | — | January | 1.8 | steam |
| hemodialyzer | H-01 | | 2020 | | |
| Reference | | | | | |

PF4 (Stago Deutschland GmbH, Dusseldorf) and TAT (Siemens Diagnostics, Marburg) was quantified by ELISA. Heparin concentration was determined by the Chromogenix Heparin Coatest Kit (antiXa activity, Haemachrom Diagnostica GmbH, Essen). WBC, RBC and platelets were counted in an ABX Pentra 60 cell counter (Axon Lab AG, Reichenbach). CD62P and CD41a (Becton Dickinson GmbH, Heidelberg) were analyzed by flow-cytometry (FacsVerse, Becton Dickinson GmbH, Heidelberg). Data acquisition and analysis were carried out by the FACSuite Software (version 1.05, Becton Dickinson GmbH, Heidelberg). In each sample 5,000 cells were recorded, which were CD41a positive and within a gate of forward (FSC) vs. side scatter (SSC) where platelets are located. FSC and SSC as well as the fluorescence data were acquired on biexponential scale. Fluorescence of the CD62P positive platelets is expressed as geometrical mean. As a positive control, 35 μM thrombin receptor activating peptide (TRAP) was incubated with each blood sample for 20 min.

Data analysis of the respective parameters was performed by two-way (balanced) ANOVA using the software Minitab release 17 (Additive GmbH, Friedrichsdorf, Germany). A statistical model was built in which the factors blood pool, materials (i.e. the different filters) and duplicate measurements were taken into account. A probability of p<0.05 was considered significant.

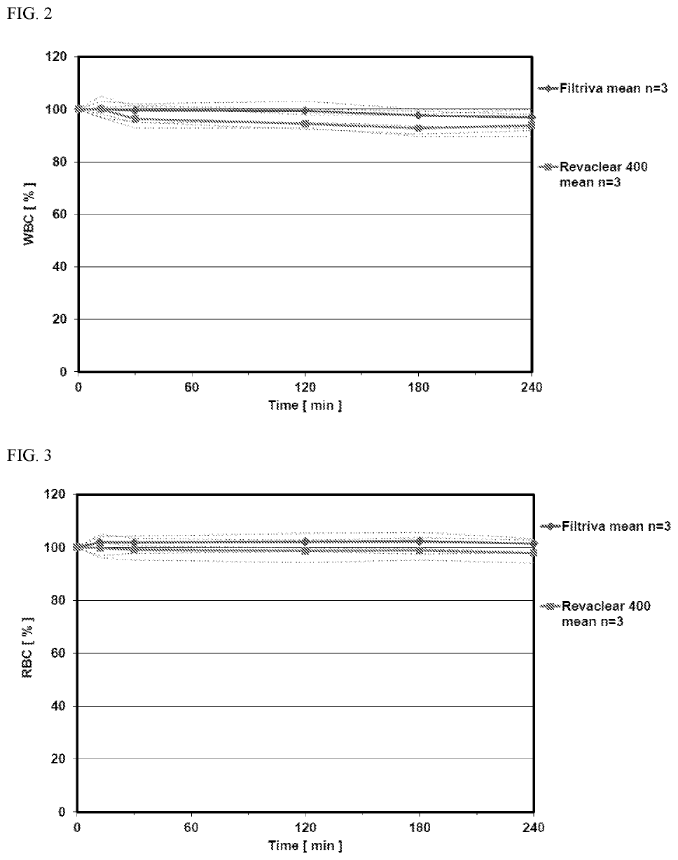

The Tables below summarize the results after 0, 12, 30, 120, 180 and 240 minutes (see also FIG. 2-8; individual experiments are shown in dotted lines).

TABLE 3
number of WBC, number of RBC and platelet count (PC)
(mean ± SD) in human blood at different time points:

| | | | | | | |
|---|---|---|---|---|---|---|
| | 0 | 12 | 30 | 120 | 180 | 240 |
| Dialyzer | min | min | min | min | min | min |

WBC [%]

| | | | | | | |
|---|---|---|---|---|---|---|
| Filtriva | 100 ± | 100 ± | 100* ± | 99* ± | 98*± | 97* ± |
| | 1 | 3 | 3 | 4 | 4 | 3 |
| Revaclear 400 | | 100 ± | 96 ± | 94 ± | 93 ± | 94 ± |
| hemodialyzer | | 4 | 4 | 3 | 4 | 5 |
| Reference | | | | | | |

RBC [%]

| | | | | | | |
|---|---|---|---|---|---|---|
| Filtriva | 100 ± | 102* ± | 102* ± | 102* ± | 102* ± | 101* ± |
| | 0 | 4 | 3 | 3 | 4 | 3 |
| Revaclear 400 | | 100 ± | 99 ± | 99 ± | 99 ± | 98 ± |
| hemodialyzer | | 3 | 3 | 4 | 3 | 3 |
| Reference | | | | | | |

PC [%]

| | | | | | | |
|---|---|---|---|---|---|---|
| Filtriva | 100 ± | 79* ± | 68* ± | 89* ± | 74* ± | 98* ± |
| | 5 | 12 | 21 | 25 | 7 | 20 |
| Revaclear 400 | | 11 ± | 3 ± | 12 ± | 18 ± | 27 ± |
| hemodialyzer | | 6 | 1 | 4 | 4 | 3 |
| Reference | | | | | | |

*means are statistically significantly different from each other (p < 0.05, read columns only)

TABLE 4
PF4 [IU/mL] in human plasma at different time points:

| | Experiment | | | | | | |
|---|---|---|---|---|---|---|---|
| | Device | 0 | 12 | 30 | 120 | 180 | 240 |
| Dialyzer | Number | min | min | min | min | min | min |
| Filtriva | 1^{st} experiment | 76 | 148 | 252 | 476 | 547 | 652 |
| | B17060053-14 | | | | | | |
| | 2^{nd} experiment | 91 | 648 | 1281 | 2082 | 2295 | 2252 |
| | B17060053-012 | | | | | | |
| | 3^{rd} experiment | 26 | 62 | <137^{#} | 195 | 206 | 231 |
| | B17060053-013 | | | | | | |
| | Mean ± SD | 64* ± | 286* ± | 767* ± | 918* ± | 1016* ± | 1045* ± |
| | | 31 | 283 | 595 | 910 | 1002 | 958 |
| Revaclear 400 | 1^{st} experiment | 214 | 1033 | 1790 | 2368 | 2277 | 2475 |
| hemodialyzer | 2^{nd} experiment | 657 | 2272 | 3399 | 4345 | 3761 | 4106 |
| Reference | 3^{rd} experiment | 50 | 973 | 2332 | 2798 | 2795 | 3348 |
| | Mean ± SD | 307 ± | 1426 ± | 2507 ± | 3170 ± | 2944 ± | 3309 ± |
| | | 281 | 662 | 733 | 933 | 679 | 738 |

^{#}value was omitted for analysis/statistics

TABLE 5
CD62P [geometrical mean of fluorescence intensity] on
platelets at different time points:

| | Experiment | | | | | | |
|---|---|---|---|---|---|---|---|
| | Device | 0 | 12 | 30 | 120 | 180 | 240 |
| Dialyzer | Number | min | min | min | min | min | min |
| Filtriva | 1^{st} experiment | 26 | 26 | 24 | 21 | 20 | 20 |
| | B17060053-014 | | | | | | |
| | 2^{nd} experiment | 21 | 34 | 34 | 49 | 63 | 72 |
| | B17060053-012 | | | | | | |
| | 3^{rd} experiment | 25 | 31 | 27 | 24 | 21 | 23 |
| | B17060053-013 | | | | | | |
| | Mean ± SD | 24 ± | 30* ± | 28* ± | 31* ± | 34 ± | 38* ± |
| | | 3 | 8 | 6 | 14 | 22 | 26 |
| Revaclear | 1^{st} experiment | 25 | 40 | 36 | 25 | 22 | 24 |
| 400 | 2^{nd} experiment | 24 | 68 | 73 | 53 | 40 | 35 |
| hem | 3^{rd} experiment | 25 | 56 | 53 | 33 | 33 | 37 |
| dialyzer | Mean ± SD | 25 ± | 55 ± | 54 ± | 37 ± | 31 ± | 32 ± |
| Refer- | | 2 | 16 | 16 | 13 | 8 | 7 |
| ence | | | | | | | |

*means are statistically significantly different from each other (p < 0.05, read columns only)

TABLE 6
TAT [ng/mL] in human plasma at different time points:

| | Experiment | | | | | | |
|---|---|---|---|---|---|---|---|
| Dial- | Device | 0 | 12 | 30 | 120 | 180 | 240 |
| yzer | Number | min | min | min | min | min | min |
| Filtriva | 1^{st} experiment | 5.0 | 5.2 | 8.2 | 11.9 | 12.7 | 14.1 |
| | B17060053- | | | | | | |
| | 014 | | | | | | |
| | 2^{nd} experiment | 8.1 | 7.9 | 7.9 | 11.1 | 12.8 | 18.7 |
| | B17060053- | | | | | | |
| | 012 | | | | | | |
| | 3^{rd} experiment | 6.6 | 7.6 | 11.1 | 9.2 | 9.4 | 12.0 |
| | B17060053- | | | | | | |
| | 013 | | | | | | |
| | Mean ± SD | 6.6 ± | 6.9 ± | 9.1* ± | 10.7* ± | 11.6* ± | 14.9* ± |
| | | 1.4 | 1.4 | 1.7 | 1.4 | 1.8 | 3.1 |
| Reva- | 1^{st} experiment | 4.6 | 5.5 | 6.4 | 8.6 | 12.3 | 17.4 |
| clear | 2^{nd} experiment | 7.6 | 6.7 | 6.1 | 10.6 | 18.0 | 29.5 |
| 400 | 3^{rd} experiment | 7.9 | 8.7 | 6.7 | 8.4 | 12.1 | 16.4 |
| hemo- | Mean ± SD | 6.7 ± | 7.0 ± | 6.4 ± | 9.2 ± | 14.1 ± | 21.1 ± |
| dialyzer | | 1.7 | 1.5 | 0.4 | 1.2 | 3.0 | 6.5 |
| Refer- | | | | | | | |
| ence | | | | | | | |

TABLE 7
Heparin [IU/mL] in human plasma at different time points:

| | Experiment | | | | | | |
|---|---|---|---|---|---|---|---|
| | Device | 0 | 12 | 30 | 120 | 180 | 240 |
| Dialyzer | Number | min | min | min | min | min | min |
| Filtriva | 1^{st} experiment | 3.9 | 3.4 | 3.4 | 3.5 | 3.4 | 3.1 |
| | B17060053-014 | | | | | | |
| | 2^{nd} experiment | 3.1 | 3.5 | 3.3 | 3.1 | 3.2 | 2.8 |
| | B17060053-012 | | | | | | |
| | 3^{rd} experiment | 3.2 | 3.2 | 3.2 | 3.3 | 3.2 | 3.3 |
| | B17060053-013 | | | | | | |
| | Mean + SD | 3.4 ± | 3.4 ± | 3.3 ± | 3.3 ± | 3.3 ± | 3.1 ± |
| | | 0.4 | 0.2 | 0.1 | 0.2 | 0.1 | 0.3 |
| Revaclear | 1^{st} experiment | 3.6 | 3.5 | 3.4 | 3.2 | 3.2 | 3.1 |
| 400 | 2^{nd} experiment | 3.5 | 3.7 | 3.6 | 3.2 | 3.1 | 3.2 |
| hemo- | 3^{rd} experiment | 3.8 | 3.1 | 2.9 | 3.1 | 3.3 | 3.8 |
| dialyzer | Mean ± SD | 3.6 ± | 3.4 ± | 3.3 ± | 3.2 ± | 3.2 ± | 3.4 ± |
| Refer- | | 0.3 | 0.3 | 0.4 | 0.1 | 0.3 | 0.5 |
| ence | | | | | | | |

*: means are statistically significantly different from each other (p < 0.05, read columns only)

White blood cell count (WBC): In hemocompatibility experiments, a drop in WBC ex vivo may indicate adhesion of these cells to the extracorporeal system. The drop in white blood cell count during the experiment is expressed as % of the initial value. Experiments performed with Q_{B}=400 mL/min caused only a very small loss of WBC for both filters, which was for Revaclear slightly higher (i.e. slightly more WBC adhesion) as compared to Filtriva. All data are >90% (see FIG. 2).

Red blood cell count (RBC): A drop in red blood cell count reflects the damage of red cells due to e.g. mechanical stress or adhesion of the cells within the experimental set up. In the experiments, RBC can be regarded as identical for Filtriva and Revaclear although statistical differences were observed (see FIG. 3).

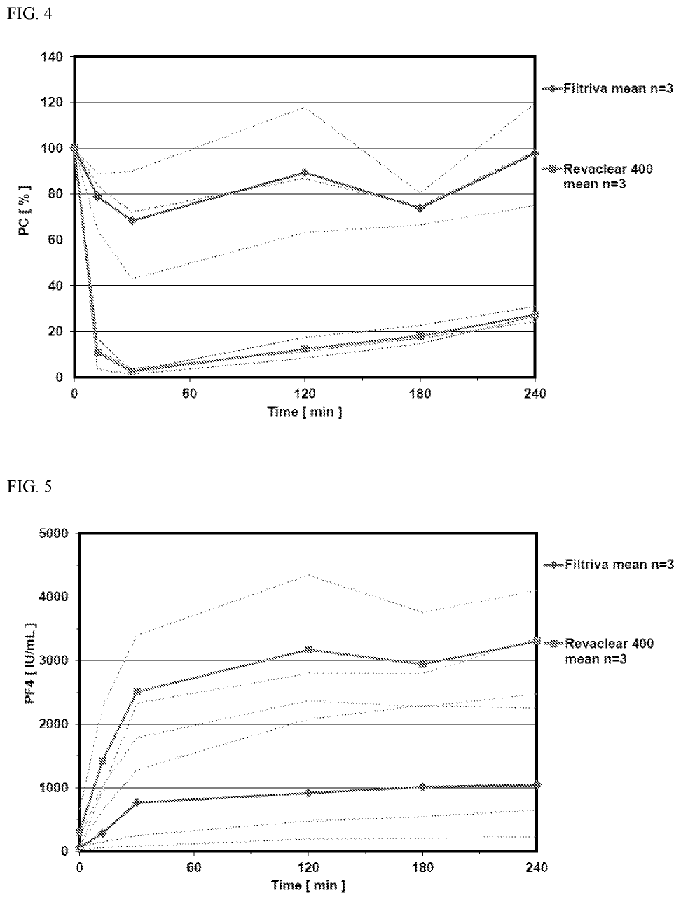

Platelet count (PC): Platelet count after 12, 30, 120, 180 and 240 min is given as percentage of the initial PC (t=0 min). Platelets adhere to hydrophobic surfaces. Therefore, all synthetic PSu and PES-based dialyzers show a pronounced drop in platelet count ex vivo which is, normally, not seen during clinical treatments. The difference between ex vivo and the clinical situation can very likely be explained by the much higher surface area to blood volume ratio in the ex vivo set-up with a blood reservoir of only 240 mL for each filter. A lower PC means stronger adhesion of platelets to the synthetic materials (membrane/tubing/housing) in the blood circuit. In the experiments, the low PC of the Revaclear 400 reference was in the range observed previously for other PSu/PES membrane containing devices. Interestingly, the Filtriva showed a significantly lower drop of platelet count as compared to the Revaclear 400 hemofilter (see FIG. 4). This observation might suggest the Filtriva membranes are more hydrophilic after Opsonix's manufacturing steps.

Platelet factor 4 (PF4): PF4 is released from α-granules of platelets upon platelet activation. It binds and inactivates heparin and thus serves as pro-coagulant. It is of note that PF4 can indicate activation of both platelets adhering to material surfaces as well as platelets remaining in blood/circulation during the experiments. Mean PF4 values increased over the whole length of the experiment for both devices, in which the Revaclear 400 dialyzer caused significantly higher PF4 values throughout as compared to the Filtriva (see FIG. 5).

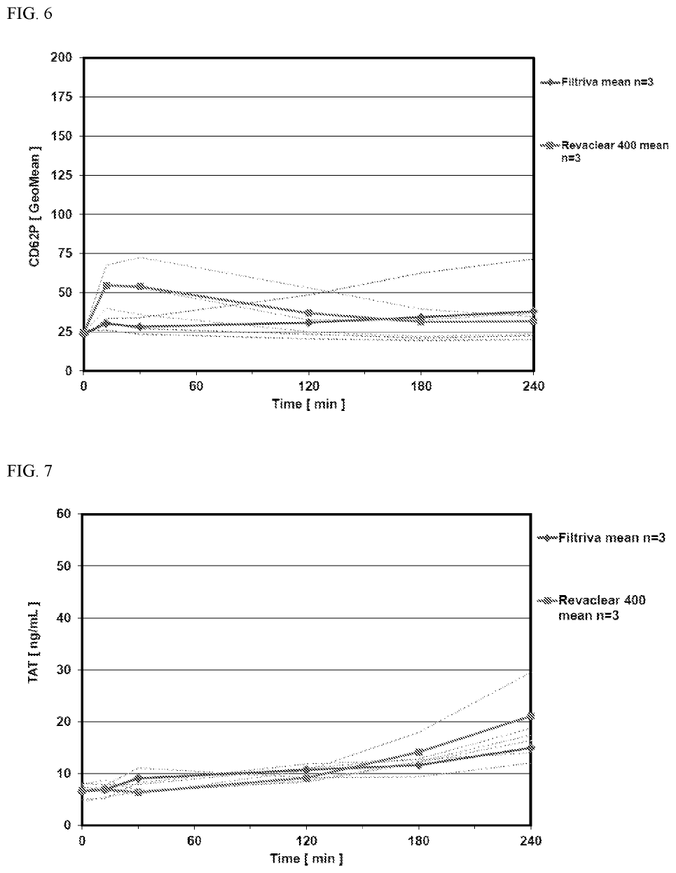

CD62P: CD62P (platelet P-selectin) is rapidly translocated from α-granules of platelets during activation and mediates interactions of activated platelets with neutrophils and monocytes. Thus, in contrast to PF4, in this experimental set-up, increased CD62P values indicate only activation of those platelets remaining in circulation. CD62P increased for Revaclear 400 till time point 12 min and decreased afterwards over time. Filtriva showed only a slight increase in CD62P till time point 240 min. Although statistical differences were observed, no biological relevance is ascribed to these observations. Indeed, mean CD62P values can be regarded as low for both filters as compared to the parallel analysis performed whereby each donors' blood was incubated with the positive control TRAP as a platelet activation stimulus (geometrical means of fluorescence intensity from TRAP-incubated samples: 1^{st} experiment: 1376, 2^{nd} experiment: 209, 3^{rd} experiment: 350). Also, expression of CD62P for both devices (see FIG. 6) is in the range of other dialyzers previously observed in the set-up.

Thrombin-Antithrombin III-Complex (TAT): Formation of the TAT complex is a very sensitive parameter for the activation of the coagulation system. Thrombin generated is quickly bound to the excess of antithrombin III in plasma. Thus, increased TAT values indicate a higher generation of thrombin and activation of the coagulation pathways. In the experiments, TAT values increased only slightly and were comparable for both filters. The differences were statistically significant from time point 30-240 min, but these differences are without biological meaning since all TAT values can be regarded as low (see FIG. 7). In the set-up only TAT values of filters >60 ng/mL are regarded as warning signals. During the coagulation process (e.g. human blood ex vivo without anticoagulant) TAT concentrations can reach values of >10,000 ng/mL, easily and within minutes.

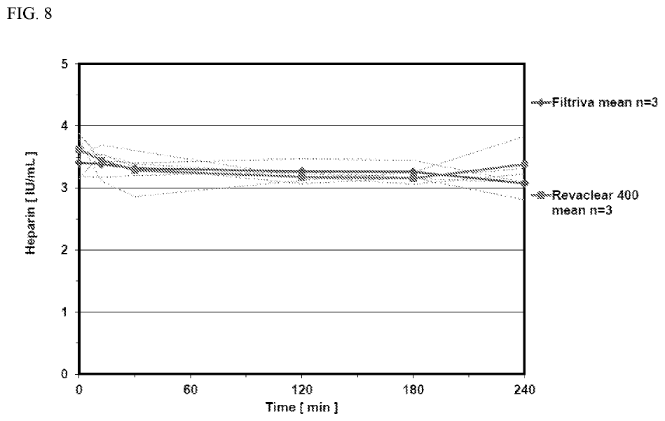

Heparin: Heparin can be bound to surfaces (positively charged surfaces, in particular) or its activity can go down during the experiment due to heparin metabolism and consumption. A lower value may indicate heparin adhesion to the filter and/or a higher consumption of heparin. Heparin was stable over the whole length of the experiment for both Filtriva™ and Revaclear™ 400 (see FIG. 8). No statistical differences were observed.

The Filtriva™ and the Revaclear™ 400 filters behaved very similar with respect to the parameters WBC count, RBC count, heparin, CD62P and TAT, but different in PC and PF4 release. Platelet loss of the Revaclear™ 400 filter was in a typical range, known from previous ex vivo studies with PES (and PSu) based filters, whereas the Filtriva™ showed a lower drop of platelet count, which is thought to be due to the modification of the lumen surface of the fibers. PF4 increased in a time dependent manner for both dialyzers, but a distinct higher increase of PF4 was observed for the Revaclear™ 400 dialyzer as compared to the Filtriva™. Mean CD62P values are considered very low for both dialyzers since the positive control showed geometrical means of fluorescence intensity >200. Mean TAT values increased slightly over time and were similar for both dialyzers, in which the Revaclear™ 400 showed a slightly higher TAT value at the end of the experiment (time point 240 min). Nevertheless, all TAT data are considered very low and in the same range as during a perfectly anticoagulated clinical treatment.

Taken together, the parameters WBC count, RBC count, PC, PF4, CD62P, TAT and heparin demonstrated, that the coating technology of the Filtriva has neither an impact on platelet activation nor on the activation of the coagulation system.

### Example 2

Recent studies have demonstrated that early administration of a combination of an antiviral such as remdesivir and glycopeptide antimicrobials, such as the antibiotics teicoplanin or dalbavancin, can prevent SARS-CoV-2 infection and transition to COVID-19. Vancomycin is the foundational member of this class of glycopeptides. The antimicrobials show both clinical longevity and a still preferential role in the therapy of methicillin-resistant Staphylococcus aureus and of susceptible Enterococcus spp.

The blood filtration devices of the invention, i.e. those with at least one component coated with the collectin MBL, are capable of depleting microbials and MAMPS from a fluid, such as blood, without the concomitant depletion of glycopeptide antibiotics (i.e. vancomycin) often seen with filters through which blood flows.

As shown in the following Example, the filters of the invention maintain adequate therapeutic level of antibiotics, less platelet loss, and broad spectrum depletion of microbials and endotoxins.

Table 8 below demonstrates that aged Filtriva™ had better clearance than Revaclear™ 400. The devices used in this example are the Filtriva™ and Revaclear™ 400 devices described above in Example 1.

The antibiotic clearance data show acceptable performance for unaged and aged Filtriva™ devices at all Qbs tested when compared to Revaclear™ 400. Rows 4 and 5 of the data in Table 8 show percentage comparisons.

TABLE 8

| | Clearance in mL/min | | | | | |
|---|---|---|---|---|---|---|
| | Fluconazole | | Gentamycin | | Vancomycin | |
| | Mean | Stdev | Mean | Stdev | Mean | Stdev |
| Flitriva, unaged N = 3 | 122 | 19 | 140 | 10 | 117 | 9 |
| Flitriva, aged, N = 3 | 119 | 10 | 136 | 7 | 115 | 9 |
| Revaclear 400, 790-00042 | 112 | 20 | 129 | 10 | 105 | 15 |
| & 790-00097, N = 6 | | | | | | |
| Flitriva, unaged vs | 8% | | 9% | | 12% | |
| Revaclear 400 | | | | | | |
| Flitriva, aged vs | 6% | | 6% | | 9% | |
| Revaclear 400 | | | | | | |
| Flitriva, aged & | 120 | 19 | 138 | 10 | 116 | 9 |
| unaged, N = 6 | | | | | | |

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

Various modifications of the invention and many further aspects thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various aspects and equivalents thereof.

### REFERENCES

All patents and publications mentioned in this specification are indicative of the level of skill of those skilled in the art to which the invention pertains. Each cited patent and publication is incorporated herein by reference in its entirety. All of the following references have been cited in this application:
1. Auriti, C. “Mannose-Binding Lectin: Biologic Characteristics and Role in the Susceptibility to Infections and Ischemia-Reperfusion Related Injury in Critically Ill Neonates,” J. Immunol. Res., Volume 2017, Article ID 7045630, 11 pages, 2017.
2. D. A. Fraser and A. J. Tenner, “Directing an appropriate immune response: the role of defense collagens and other soluble pattern recognition molecules,” Current Drug Targets, vol. 9, no. 2, pp. 113-122, 2008.
3. J. Nadesalingam, A. W. Dodds, K. B. M. Reid, and N. Palaniyar, “Mannose-binding lectin recognizes peptidoglycan via the N-acetyl glucosamine moiety, and inhibits ligand-induced proinflammatory effect and promotes chemokine production by macrophages,” The Journal of Immunology, vol. 175, no. 3, pp. 1785-1794, 2005.
4. D. C. Kilpatrick, “Mannan-binding lectin: clinical significance and applications,” Biochimica et Biophysica Acta, vol. 1572, no. 2-3, pp. 401-413, 2002.
5. N. J. Klein, “Mannose-binding lectin: do we need it?” Molecular Immunology, vol. 42, no. 8, pp. 919-924, 2005.
6. K. Takahashi, W. K. E. Ip, I. C. Michelow, and R. A. B. Ezekowitz, “The mannose-binding lectin: a prototypic pattern recognition molecule,” Current Opinion in Immunology, vol. 18, no. 1, pp. 16-23, 2006.
7. J.-L. Casanova and L. Abel, “Human mannose-binding lectin in immunity: friend, foe, or both?” Journal of Experimental Medicine, vol. 199, no. 10, pp. 1295-1299, 2004.
8. A. S. Grumach, M. E. Ceccon, R. Rutz, A. Fertig, and M. Kirschfink, “Complement profile in neonates of different gestational ages,” Scandinavian Journal of Immunology, vol. 79, no. 4, pp. 276-281, 2014.
9. O. Neth, D. L. Jack, M. Johnson, N. J. Klein, and M. W. Turner, “Enhancement of complement activation and opsonophagocytosis by complexes of mannose-binding lectin with mannose-binding lectin-associated serine protease after binding to Staphylococcus aureus,” Journal of Immunology, vol. 169, no. 8, pp. 4430-4436, 2002.
10. S. Thiel, T. Vorup-Jensen, C. M. Stover et al., “A second serine protease associated with mannan-binding lectin that activates complement,” Nature, vol. 386, no. 6624, pp. 506-510, 1997.
11. M. Matsushita and T. Fujita, “Activation of the classical complement pathway by mannose-binding protein in association with a novel C1s-like serine protease,” The Journal of Experimental Medicine, vol. 176, no. 6, pp. 1497-1502, 1992.
12. M. Matsushita and T. Fujita, “Cleavage of the third component of complement (C3) by mannose-binding protein-associated serine protease (MASP) with subsequent complement activation,” Immunobiology, vol. 194, no. 4-5, pp. 443-448, 1995.

What is claimed is:

1. A substrate comprising at least one surface coated with one or more collectins.

2. The substrate of claim 1, wherein the substrate is selected from the group consisting of a bead, a particle, a filter, a fiber, a screen, a mesh, a membrane, a semi-permeable membrane, a permeable membrane, a cartridge, a tube, a hollow fiber, a cell-culture scaffold, a cell-culture plate, a channel, a gold particle, a magnetic material, a needle, a catheter, a syringe, a medical or dental appliance, a medical or dental implant, a medical component, a dialyzer, a hemodialyzer, a dipstick, a test strip, a filtration device, a filtration device membrane, a hollow fiber cartridge, a microfluidic device, a mixing element, a component of a medical device, a component of a therapeutic device, a filtration device, an oxygenation device, an extracorporeal device, a dialysis device, an infusion device, a drainage device, and a pump.

3. The substrate of claim 2, wherein the substrate is a filter.

4. The substrate of claim 2, wherein the substrate is a filter of an extracorporeal device.

5. The substrate of any one of claims 1-4, wherein the collectin is selected from the group consisting of (i) mannose-binding lectin (MBL), (ii) surfactant protein A (SP-A), (iii) surfactant protein D (SP-D), (iv) collectin liver 1 (CL-L1), (v) collectin placenta 1 (CL-P1), (vi) conglutinin collectin of 43 kDa (CL-43), (vii) collectin of 46 kDa (CL-46), (viii) collectin kidney 1 (CL-K1), and (ix) conglutinin.

6. The substrate of claim 5, wherein the collectin is (i) a naturally-occurring MBL, (ii) a truncated form of naturally-occurring MBL, (iii) an engineered form of MBL, or (iv) a sequence variant of (i), (ii) or (iii).

7. The substrate of claim 6, wherein the collectin is a naturally-occurring MBL as set forth in SEQ ID NO:1 or a sequence variant having at least 80% sequence identity with SEQ ID NO:1 that retains the ability to reduce platelet activation in blood.

8. The substrate of claim 6, wherein the collectin is a truncated form of naturally-occurring MBL as set forth in any one of SEQ ID NOs:2-5 or a sequence variant having at least 80% sequence identity with any one of SEQ ID NOs:2-5 that retains the ability to reduce platelet activation in blood.

9. The substrate of claim 6, wherein the collectin is an engineered form of MBL comprising a fusion protein of an MBL of any one of SEQ ID NOs:1-5 and an Fe domain of an IgG antibody.

10. The substrate of claim 9, wherein the fusion protein is a FcMBL protein set forth in any one of SEQ ID NOs:6-9, or a sequence variant having at least 80% sequence identity with any one of SEQ ID NOs:6-9 that retains the ability to reduce platelet activation in blood.

11. The substrate of claim 1, wherein the one or more collectins are immobilized on the at least one surface via a non-specific interaction between the collectins and the surface.

12. The substrate of claim 1, wherein the one or more collectins are immobilized on the at least one surface via a specific interaction between the collectins and the surface.

13. The substrate of claim 12, wherein the specific interaction is a covalent amide bond.

14. The substrate of claim 13, wherein 3-(3-dimethylaminopropyl)carbodiimide (EDC) is used to produce the covalent amide bond.

15. The substrate of claim 1, wherein prior to coating with the one or more collectins, the at least one surface is fabricated of or coated with one or more of polydimethylsiloxane, polyimide, polyethylene terephthalate, polymethylmethacrylate, polyurethane, polyvinylchloride, polystyrene polysulfone, polycarbonate, polymethylpentene, polypropylene, a polyvinylidine fluoride, polysilicon, polytetrafluoroethylene, polysulfone, acrylonitrile butadiene styrene, polyacrylonitrile, polybutadiene, poly(butylene terephthalate), poly(ether sulfone), poly(ether ether ketones), poly(ethylene glycol), styrene-acrylonitrile resin, poly(trimethylene terephthalate), polyvinyl butyral, polyvinylidenedifluoride, poly(vinyl pyrrolidone), and any combination thereof.

16. The substrate of claim 1, wherein prior to coating with the one or more collectins, the at least one surface is functionalized to include at least one molecule exhibiting binding specificity for the collectin.

17. The substrate of claim 1, wherein the at least one surface is further coated with an anticoagulant.

18. The substrate of claim 17, wherein the anticoagulant is selected from the group consisting of heparin, warfarin, rivaroxaban, dabigatran, apixaban, edoxaban, enoxaparin, or and fondaparinux.

19. A method for reducing platelet activation in a subject undergoing a medical procedure, said method comprising conducting a medical procedure on the subject using a medical device comprising at least one substrate of claim 1.

20. A method for reducing platelet activation in a subject undergoing an extracorporeal therapy, said method comprising conducting the extracorporeal therapy on the subject using a device comprising at least one substrate of claim 1.

21. A method for reducing platelet activation in blood of a subject undergoing a medical procedure, said method comprising contacting blood of the subject using a device comprising at least one substrate of claim 1.

22. The method of claim 21, wherein the medical procedure is oxygenation of the blood, or removal of carbon dioxide from the blood, or both.

23. A method for reducing antibiotic loss from blood of a subject undergoing a medical procedure, said method comprising conducting a medical procedure on the subject using a medical device comprising at least one substrate of claim 1.

24. A method for reducing antibiotic loss from blood of a subject undergoing an extracorporeal therapy, said method comprising conducting the extracorporeal therapy on the subject using a device comprising at least one substrate of claim 1.

25. A method for reducing ficolin-2 loss from blood of a subject undergoing a medical procedure, said method comprising conducting a medical procedure on the subject using a medical device comprising at least one substrate of claim 1.

26. A method for reducing ficolin-2 loss from blood of a subject undergoing an extracorporeal therapy, said method comprising conducting the extracorporeal therapy on the subject using a device comprising at least one substrate of claim 1.


SEQUENCE LISTING (17 sequences)

SEQ ID NO: 1 - protein, 248 aa, Homo sapiens
MSLFPSLPLLLLSMVAASYSETVTCEDAQKTCPAVIACSSPGINGFPGKDGRDGTKGEKGEPGQGLRGLQGPPGKLGPPGNPGPSGSPGPKGQKGDPGKSPDGDSSLAASERKALQTEMARIKKWLTFSLGKQVGNKFFLTNGEIMTFEKVKALCVKFQASVATPRNAAENGAIQNLIKEEAFLGITDEKTEGQFVDLTGNRLTYTNWNEGEPNNAGSDEDCVLLLKNGQWNDVPCSTSHLAVCEFPI

SEQ ID NO: 2 - protein, 228 aa, Homo sapiens
ETVTCEDAQKTCPAVIACSSPGINGFPGKDGRDGTKGEKGEPGQGLRGLQGPPGKLGPPGNPGPSGSPGPKGQKGDPGKSPDGDSSLAASERKALQTEMARIKKWLTFSLGKQVGNKFFLTNGEIMTFEKVKALCVKFQASVATPRNAAENGAIQNLIKEEAFLGITDEKTEGQFVDLTGNRLTYTNWNEGEPNNAGSDEDCVLLLKNGQWNDVPCSTSHLAVCEFPI

SEQ ID NO: 3 - protein, 141 aa, Homo sapiens
AASERKALQTEMARIKKWLTFSLGKQVGNKFFLTNGEIMTFEKVKALCVKFQASVATPRNAAENGAIQNLIKEEAFLGITDEKTEGQFVDLTGNRLTYTNWNEGEPNNAGSDEDCVLLLKNGQWNDVPCSTSHLAVCEFPI

SEQ ID NO: 4 - protein, 115 aa, Homo sapiens
VGNKFFLTNGEIMTFEKVKALCVKFQASVATPRNAAENGAIQNLIKEEAFLGITDEKTEGQFVDLTGNRLTYTNWNEGEPNNAGSDEDCVLLLKNGQWNDVPCSTSHLAVCEFPI

SEQ ID NO: 5 - protein, 148 aa, Homo sapiens
PDGDSSLAASERKALQTEMARIKKWLTFSLGKQVGNKFFLTNGEIMTFEKVKALCVKFQASVATPRNAAENGAIQNLIKEEAFLGITDEKTEGQFVDLTGNRLTYTNWNEGEPNNAGSDEDCVLLLKNGQWNDVPCSTSHLAVCEFPI

SEQ ID NO: 6 - protein, 381 aa, Artificial Sequence
  Recombinantly-produced fusion protein
EPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGAPDGDSSLAASERKALQTEMARIKKWLTFSLGKQVGNKFFLTNGEINITFEKVKALCVKFQASVATPRNAAENGAIQNLIKEEAFLGITDEKTEGQFVDLTGNRLTYTNWNEGEPNNAGSDEDCVLLLKNGQWNDVPCSTSHLAVCEFPI

SEQ ID NO: 7 - protein, 383 aa, Artificial Sequence
  Recombinantly-produced fusion protein
AKTEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGAPDGDSSLAASERKALQTEMARIKKWLTFSLGKQVGNKFFLTNGEIMTFEKVKALCVKFQASVATPRNAAENGAIQNLIKEEAFLGITDEKTEGQFVDLTGNRLTYTNWNEGEPNNAGSDEDCVLLLKNGQWNDVPCSTSHLAVCEFPI

SEQ ID NO: 8 - protein, 352 aa, Artificial Sequence
  Recombinantly-produced fusion protein
EPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGATSKQVGNKFFLTNGEEVITFEKVKALCVKFQASVATPRNAAENGAIQNLIKEEAFLGITDEKTEGQFVDLTGNRLTYTNWNEGEPNNAGSDEDCVLLLKNGQWNDVPCSTSHLAVCEFPI

SEQ ID NO: 9 - protein, 383 aa, Artificial Sequence
  Recombinantly-produced fusion protein
AKTEPKSSDKTHTCPPCPAPELLGGPSVFLFPPKPKDTLMISRTPEVTCVVVDVSHEDPEVKFNWYVDGVEVHNAKTKPREEQYDSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGAPDGDSSLAASERKALQTEMARIKKWLTFSLGKQVGNKFFLTNGEIMTFEKVKALCVKFQASVATPRNAAENGAIQNLIKEEAFLGITDEKTEGQFVDLTGNRLTYTNWNEGEPNNAGSDEDCVLLLKNGQWNDVPCSTSHLAVCEFPI

SEQ ID NO: 10 - protein, 10 aa, Homo sapiens
NEGEPNNAGS

SEQ ID NO: 11 - protein, 15 aa, Homo sapiens
LLLKNGQWNDVPCST

SEQ ID NO: 12 - protein, 232 aa, Homo sapiens
EPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGA

SEQ ID NO: 13 - protein, 278 aa, Homo sapiens [sequence omitted]

SEQ ID NO: 14 - protein, 375 aa, Homo sapiens
MLLFLLSALVLLTQPLGYLEAEMKTYSHRTMPSACTLVMCSSVESGLPGRDGRDGREGPRGEKGDPGLPGAAGQAGMPGQAGPVGPKGDNGSVGEPGPKGDTGPSGPPGPPGVPGPAGREGALGKQGNIGPQGKPGPKGEAGPKGEVGAPGMQGSAGARGLAGPKGERGVPGERGVPGNTGAAGSAGAMGPQGSPGARGPPGLKGDKGIPGDKGAKGESGLPDVASLRQQVEALQGQVQHLQAAFSQYKKVELFPNGQSVGEKIFKTAGFVKPFTEAQLLCTQAGGQLASPRSAAENAALQQLVVAKNEAAFLSMTDSKTEGKFTYPTGESLVYSNWAPGEPNDDGGSEDCVEIFTNGKWNDRACGEKRLVVCEF

SEQ ID NO: 15 - protein, 277 aa, Homo sapiens
MNGFASLLRRNQFILLVLFLLQIQSLGLDIDSRPTAEVCATHTISPGPKGDDGEKGDPGEEGKHGKVGRMGPKGIKGELGDMGDQGNIGKTGPIGKKGDKGEKGLLGIPGEKGKAGTVCDCGRYRKFVGQLDISIARLKTSMKFVKNVIAGIRETEEKFYYIVQEEKNYRESLTHCRIRGGMLAMPKDEAANTLIADYVAKSGFFRVFIGVNDLEREGQYMFTDNTPLQNYSNWNEGEPSDPYGHEDCVEMLSSGRWNDTECHLTMYFVCEFIKKKK

SEQ ID NO: 16 - protein, 742 aa, Homo sapiens
MKDDFAEEEEVQSFGYKRFGIQEGTQCTKCKNNWALKFSIILLYILCALLTITVAILGYKVVEKMDNVTGGMETSRQTYDDKLTAVESDLKKLGDQTGKKAISTNSELSTFRSDILDLRQQLREITEKTSKNKDTLEKLQASGDALVDRQSQLKETLENNSFLITTVNKTLQAYNGYVTNLQQDTSVLQGNLQNQMYSHNVVIMNLNNLNLTQVQQRNLITNLQRSVDDTSQAIQRIKNDFQNLQQVFLQAKKDTDWLKEKVQSLQTLAANNSALAKANNDTLEDMNSQLNSFTGQMENITTISQANEQNLKDLQDLHKDAENRTAIKFNQLEERFQLFETDIVNIISNISYTAHHLRTLTSNLNEVRTTCTDTLTKHTDDLTSLNNTLANIRLDSVSLRMQQDLMRSRLDTEVANLSVIMEEMKLVDSKHGQLIKNFTILQGPPGPRGPRGDRGSQGPPGPTGNKGQKGEKGEPGPPGPAGERGPIGPAGPPGERGGKGSKGSQGPKGSRGSPGKPGPQGPSGDPGPPGPPGKEGLPGPQGPPGFQGLQGTVGEPGVPGPRGLPGLPGVPGMPGPKGPPGPPGPSGAVVPLALQNEPTPAPEDNGCPPHWKNFTDKCYYFSVEKEIFEDAKLFCEDKSSHLVFINTREEQQWIKKQMVGRESHWIGLTDSERENEWKWLDGTSPDYKNWKAGQPDNWGHGHGPGEDCAGLIYAGQWNDFQCEDVNNFICEKDRETVLSSAL

SEQ ID NO: 17 - protein, 271 aa, Homo sapiens
MRGNLALVGVLISLAFLSLLPSGHPQPAGDDACSVQILVPGLKGDAGEKGDKGAPGRPGRVGPTGEKGDMGDKGQKGSVGRHGKIGPIGSKGEKGDSGDIGPPGPNGEPGLPCECSQLRKAIGEMDNQVSQLTSELKFIKNAVAGVRETESKIYLLVKEEKRYADAQLSCQGRGGTLSMPKDEAANGLMAAYLAQAGLARVFIGINDLEKEGAFVYSDHSPMRTFNKWRSGEPNNAYDEEDCVEMVASGGWNDVACHTTMYFMCEFDKENM